SEQUENCE LISTING (14 sequences)

SEQ ID NO: 1 - DNA, 10719 bp, Homo sapiens
tggagacagaatggaggtgctgccggactcggaaatggggtccaagggtagccaaggatggctgcagcttcatatgatcagttgttaaagcaagttgaggcactgaagatggagaactcaaatcttcgacaagagctagaagataattccaatcatcttacaaaactggaaactgaggcatctaatatgaaggaagtacttaaacaactacaaggaagtattgaagatgaagctatggcttcttctggacagattgatttattagagcgtcttaaagagcttaacttagatagcagtaatttccctggagtaaaactgcggtcaaaaatgtccctccgttcttatggaagccgggaaggatctgtatcaagccgttctggagagtgcagtcctgttcctatgggttcatttccaagaagagggtttgtaaatggaagcagagaaagtactggatatttagaagaacttgagaaagagaggtcattgcttcttgctgatcttgacaaagaagaaaaggaaaaagactggtattacgctcaacttcagaatctcactaaaagaatagatagtcttcctttaactgaaaatttttccttacaaacagatatgaccagaaggcaattggaatatgaagcaaggcaaatcagagttgcgatggaagaacaactaggtacctgccaggatatggaaaaacgagcacagcgaagaatagccagaattcagcaaatcgaaaaggacatacttcgtatacgacagcttttacagtcccaagcaacagaagcagagaggtcatctcagaacaagcatgaaaccggctcacatgatgctgagcggcagaatgaaggtcaaggagtgggagaaatcaacatggcaacttctggtaatggtcagggttcaactacacgaatggaccatgaaacagccagtgttttgagttctagtagcacacactctgcacctcgaaggctgacaagtcatctgggaaccaaggtggaaatggtgtattcattgttgtcaatgcttggtactcatgataaggatgatatgtcgcgaactttgctagctatgtctagctcccaagacagctgtatatccatgcgacagtctggatgtcttcctctcctcatccagcttttacatggcaatgacaaagactctgtattgttgggaaattcccggggcagtaaagaggctcgggccagggccagtgcagcactccacaacatcattcactcacagcctgatgacaagagaggcaggcgtgaaatccgagtccttcatcttttggaacagatacgcgcttactgtgaaacctgttgggagtggcaggaagctcatgaaccaggcatggaccaggacaaaaatccaatgccagctcctgttgaacatcagatctgtcctgctgtgtgtgttctaatgaaactttcatttgatgaagagcatagacatgcaatgaatgaactagggggactacaggccattgcagaattattgcaagtggactgtgaaatgtatgggcttactaatgaccactacagtattacactaagacgatatgctggaatggctttgacaaacttgacttttggagatgtagccaacaaggctacgctatgctctatgaaaggctgcatgagagcacttgtggcccaactaaaatctgaaagtgaagacttacagcaggttattgcgagtgttttgaggaatttgtcttggcgagcagatgtaaatagtaaaaagacgttgcgagaagttggaagtgtgaaagcattgatggaatgtgctttagaagttaaaaaggaatcaaccctcaaaagcgtattgagtgccttatggaatttgtcagcacattgcactgagaataaagctgatatatgtgctgtagatggtgcacttgcatttttggttggcactcttacttaccggagccagacaaacactttagccattattgaaagtggaggtgggatattacggaatgtgtccagcttgatagctacaaatgaggaccacaggcaaatcctaagagagaacaactgtctacaaactttattacaacacttaaaatctcatagtttgacaatagtcagtaatgcatgtggaactttgtggaatctctcagcaagaaatcctaaagaccaggaagcattatgggacatgggggcagttagcatgctcaagaacctcattcattcaaagcacaaaatgattgctatgggaagtgctgcagctttaaggaatctcatggcaaataggcctgcgaagtacaaggatgccaatattatgtctcctggctcaagcttgccatctcttcatgttaggaaacaaaaagccctagaagcagaattagatgctcagcacttatcagaaacttttgacaatatagacaatttaagtcccaaggcatctcatcgtagtaagcagagacacaagcaaagtctctatggtgattatgtttttgacaccaatcgacatgatgataataggtcagacaattttaatactggcaacatgactgtcctttcaccatatttgaatactacagtgttacccagctcctcttcatcaagaggaagcttagatagttctcgttctgaaaaagatagaagtttggagagagaacgcggaattggtctaggcaactaccatccagcaacagaaaatccaggaacttcttcaaagcgaggtttgcagatctccaccactgcagcccagattgccaaagtcatggaagaagtgtcagccattcatacctctcaggaagacagaagttctgggtctaccactgaattacattgtgtgacagatgagagaaatgcacttagaagaagctctgctgcccatacacattcaaacacttacaatttcactaagtcggaaaattcaaataggacatgttctatgccttatgccaaattagaatacaagagatcttcaaatgatagtttaaatagtgtcagtagtagtgatggttatggtaaaagaggtcaaatgaaaccctcgattgaatcctattctgaagatgatgaaagtaagttttgcagttatggtcaatacccagccgacctagcccataaaatacatagtgcaaatcatatggatgataatgatggagaactagatacaccaataaattatagtcttaaatattcagatgagcagttgaactctggaaggcaaagtccttcacagaatgaaagatgggcaagacccaaacacataatagaagatgaaataaaacaaagtgagcaaagacaatcaaggaatcaaagtacaacttatcctgtttatactgagagcactgatgataaacacctcaagttccaaccacattttggacagcaggaatgtgtttctccatacaggtcacggggagccaatggttcagaaacaaatcgagtgggttctaatcatggaattaatcaaaatgtaagccagtctttgtgtcaagaagatgactatgaagatgataagcctaccaattatagtgaacgttactctgaagaagaacagcatgaagaagaagagagaccaacaaattatagcataaaatataatgaagagaaacgtcatgtggatcagcctattgattatagtttaaaatatgccacagatattccttcatcacagaaacagtcattttcattctcaaagagttcatctggacaaagcagtaaaaccgaacatatgtcttcaagcagtgagaatacgtccacaccttcatctaatgccaagaggcagaatcagctccatccaagttctgcacagagtagaagtggtcagcctcaaaaggctgccacttgcaaagtttcttctattaaccaagaaacaatacagacttattgtgtagaagatactccaatatgtttttcaagatgtagttcattatcatctttgtcatcagctgaagatgaaataggatgtaatcagacgacacaggaagcagattctgctaataccctgcaaatagcagaaataaaagaaaagattggaactaggtcagctgaagatcctgtgagcgaagttccagcagtgtcacagcaccctagaaccaaatccagcagactgcagggttctagtttatcttcagaatcagccaggcacaaagctgttgaattttcttcaggagcgaaatctccctccaaaagtggtgctcagacacccaaaagtccacctgaacactatgttcaggagaccccactcatgtttagcagatgtacttctgtcagttcacttgatagttttgagagtcgttcgattgccagctccgttcagagtgaaccatgcagtggaatggtaagtggcattataagccccagtgatcttccagatagccctggacaaaccatgccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacggaaagtactccagatggattttcttgttcatccagcctgagtgctctgagcctcgatgagccatttatacagaaagatgtggaattaagaataatgcctccagttcaggaaaatgacaatgggaatgaaacagaatcagagcagcctaaagaatcaaatgaaaaccaagagaaagaggcagaaaaaactattgattctgaaaaggacctattagatgattcagatgatgatgatattgaaatactagaagaatgtattatttctgccatgccaacaaagtcatcacgtaaagcaaaaaagccagcccagactgcttcaaaattacctccacctgtggcaaggaaaccaagtcagctgcctgtgtacaaacttctaccatcacaaaacaggttgcaaccccaaaagcatgttagttttacaccgggggatgatatgccacgggtgtattgtgttgaagggacacctataaacttttccacagctacatctctaagtgatctaacaatcgaatcccctccaaatgagttagctgctggagaaggagttagaggaggggcacagtcaggtgaatttgaaaaacgagataccattcctacagaaggcagaagtacagatgaggctcaaggaggaaaaacctcatctgtaaccatacctgaattggatgacaataaagcagaggaaggtgatattcttgcagaatgcattaattctgctatgcccaaagggaaaagtcacaagcctttccgtgtgaaaaagataatggaccaggtccagcaagcatctgcgtcttcttctgcacccaacaaaaatcagttagatggtaagaaaaagaaaccaacttcaccagtaaaacctataccacaaaatactgaatataggacacgtgtaagaaaaaatgcagactcaaaaaataatttaaatgctgagagagttttctcagacaacaaagattcaaagaaacagaatttgaaaaataattccaaggtcttcaatgataagctcccaaataatgaagatagagtcagaggaagttttgcttttgattcacctcatcattacacgcctattgaaggaactccttactgtttttcacgaaatgattctttgagttctctagattttgatgatgatgatgttgacctttccagggaaaaggctgaattaagaaaggcaaaagaaaataaggaatcagaggctaaagttaccagccacacagaactaacctccaaccaacaatcagctaataagacacaagctattgcaaagcagccaataaatcgaggtcagcctaaacccatacttcagaaacaatccacttttccccagtcatccaaagacataccagacagaggggcagcaactgatgaaaagttacagaattttgctattgaaaatactccggtttgcttttctcataattcctctctgagttctctcagtgacattgaccaagaaaacaacaataaagaaaatgaacctatcaaagagactgagccccctgactcacagggagaaccaagtaaacctcaagcatcaggctatgctcctaaatcatttcatgttgaagataccccagtttgtttctcaagaaacagttctctcagttctcttagtattgactctgaagatgacctgttgcaggaatgtataagctccgcaatgccaaaaaagaaaaagccttcaagactcaagggtgataatgaaaaacatagtcccagaaatatgggtggcatattaggtgaagatctgacacttgatttgaaagatatacagagaccagattcagaacatggtctatcccctgattcagaaaattttgattggaaagctattcaggaaggtgcaaattccatagtaagtagtttacatcaagctgctgctgctgcatgtttatctagacaagcttcgtctgattcagattccatcctttccctgaaatcaggaatctctctgggatcaccatttcatcttacacctgatcaagaagaaaaaccctttacaagtaataaaggcccacgaattctaaaaccaggggagaaaagtacattggaaactaaaaagatagaatctgaaagtaaaggaatcaaaggaggaaaaaaagtttataaaagtttgattactggaaaagttcgatctaattcagaaatttcaggccaaatgaaacagccccttcaagcaaacatgccttcaatctctcgaggcaggacaatgattcatattccaggagttcgaaatagctcctcaagtacaagtcctgtttctaaaaaaggcccaccccttaagactccagcctccaaaagccctagtgaaggtcaaacagccaccacttctcctagaggagccaagccatctgtgaaatcagaattaagccctgttgccaggcagacatcccaaataggtgggtcaagtaaagcaccttctagatcaggatctagagattcgaccccttcaagacctgcccagcaaccattaagtagacctatacagtctcctggccgaaactcaatttcccctggtagaaatggaataagtcctcctaacaaattatctcaacttccaaggacatcatcccctagtactgcttcaactaagtcctcaggttctggaaaaatgtcatatacatctccaggtagacagatgagccaacagaaccttaccaaacaaacaggtttatccaagaatgccagtagtattccaagaagtgagtctgcctccaaaggactaaatcagatgaataatggtaatggagccaataaaaaggtagaactttctagaatgtcttcaactaaatcaagtggaagtgaatctgatagatcagaaagacctgtattagtacgccagtcaactttcatcaaagaagctccaagcccaaccttaagaagaaaattggaggaatctgcttcatttgaatctctttctccatcatctagaccagcttctcccactaggtcccaggcacaaactccagttttaagtccttcccttcctgatatgtctctatccacacattcgtctgttcaggctggtggatggcgaaaactcccacctaatctcagtcccactatagagtataatgatggaagaccagcaaagcgccatgatattgcacggtctcattctgaaagtccttctagacttccaatcaataggtcaggaacctggaaacgtgagcacagcaaacattcatcatcccttcctcgagtaagcacttggagaagaactggaagttcatcttcaattctttctgcttcatcagaatccagtgaaaaagcaaaaagtgaggatgaaaaacatgtgaactctatttcaggaaccaaacaaagtaaagaaaaccaagtatccgcaaaaggaacatggagaaaaataaaagaaaatgaattttctcccacaaatagtacttctcagaccgtttcctcaggtgctacaaatggtgctgaatcaaagactctaatttatcaaatggcacctgctgtttctaaaacagaggatgtttgggtgagaattgaggactgtcccattaacaatcctagatctggaagatctcccacaggtaatactcccccggtgattgacagtgtttcagaaaaggcaaatccaaacattaaagattcaaaagataatcaggcaaaacaaaatgtgggtaatggcagtgttcccatgcgtaccgtgggtttggaaaatcgcctgaactcctttattcaggtggatgcccctgaccaaaaaggaactgagataaaaccaggacaaaataatcctgtccctgtatcagagactaatgaaagttctatagtggaacgtaccccattcagttctagcagctcaagcaaacacagttcacctagtgggactgttgctgccagagtgactccttttaattacaacccaagccctaggaaaagcagcgcagatagcacttcagctcggccatctcagatcccaactccagtgaataacaacacaaagaagcgagattccaaaactgacagcacagaatccagtggaacccaaagtcctaagcgccattctgggtcttaccttgtgacatctgtttaaaagagaggaagaatgaaactaagaaaattctatgttaattacaactgctatatagacattttgtttcaaatgaaactttaaaagactgaaaaattttgtaaataggtttgattcttgttagagggtttttgttctggaagccatatttgatagtatactttgtcttcactggtcttattttgggaggcactcttgatggttaggaaaaaaatagtaaagccaagtatgtttgtacagtatgttttacatgtatttaaagtagcatcccatcccaacttcctttaattattgcttgtcttaaaataatgaacactacagatagaaaatatgatatattgctgttatcaatcatttctagattataaactgactaaacttacatcagggaaaaattggtatttatgcaaaaaaaaatgtttttgtccttgtgagtccatctaacatcataattaatcatgtggctgtgaaattcacagtaatatggttcccgatgaacaagtttacccagcctgctttgctttactgcatgaatgaaactgatggttcaatttcagaagtaatgattaacagttatgtggtcacatgatgtgcatagagatagctacagtgtaataatttacactattttgtgctccaaacaaaacaaaaatctgtgtaactgtaaaacattgaatgaaactattttacctgaactagattttatctgaaagtaggtagaatttttgctatgctgtaatttgttgtatattctggtatttgaggtgagatggctgctcttttattaatgagacatgaattgtgtctcaacagaaactaaatgaacatttcagaataaattattgctgtatgtaaactgttactgaaattggtatttgtttgaagggtcttgtttcacatttgtattaataattgtttaaaatgcctcttttaaaagcttatataaatttttttcttcagcttctatgcattaagagtaaaattcctcttactgtaataaaaacaattgaagaagactgttgccacttaaccattccatgcgttggcacttatctattcctgaaatttcttttatgtgattagctcatcttgatttttaatatttttccacttaaacttttttttcttactccactggagctcagtaaaagtaaattcatgtaatagcaatgcaagcagcctagcacagactaagcattgagcataataggcccacataatttcctctttcttaatattatagaattctgtacttgaaattgattcttagacattgcagtctcttcgaggctttacagtgtaaactgtcttgccccttcatcttcttgttgcaactgggtctgacatgaacactttttatcaccctgtatgttagggcaagatctcagcagtgaagtataatcagcactttgccatgctcagaaaattcaaatcacatggaactttagaggtagatttaatacgattaagatattcagaagtatattttagaatccctgcctgttaaggaaactttatttgtggtaggtacagttctggggtacatgttaagtgtccccttatacagtggagggaagtcttccttcctgaaggaaaataaactgacacttattaactaagataatttacttaatatatcttccctgatttgttttaaaagatcagagggtgactgatgatacatgcatacatatttgttgaataaatgaaaatttatttttagtgataagattcatacactctgtatttggggagggaaaacctttttaagcatggtggggcactcagataggagtgaatacacctacctggtgccttgaaaatcacatcaagtagttaattatctaccccttacctgtgtttataacttccaggtaatgagaatgattttttttaaagctaaaatgccagtaaataaaagtgctatgacttgagctaagatatttgactccaatgcctgtactgtgtctactgcaccactttgtaaacacttcaatttactatctttgaaatgattgacctttaaatttttgccaaatgttatctgaaattgtctatgaataccatctacttctgttgttttcccaggcttccataaacaatggagatacatgcaaaaaaaaaaaaaaaaaa

SEQ ID NO: 2 - DNA, 648 bp, Homo sapiens
actaggaaaactgtaacaataagagtggagatagctgtcagcaacttttgtgagggtgtgctacagggtgtagagcactgtgaagtctctacatgagtgaagtcatgatatgatcctttgagagcctttagccgccgcagaacagcagtctggctatttagatagaacaacttgattttaagataaaagaactgtctatgtagcatttatgcatttttcttaagcgtcgatggaggagtttgtaaatgaagtacagttcattacgatacacgtctgcagtcaactggaattttcatgattgaattttgtaaggtattttgaaataatttttcatataaaggtgagtttgtattaaaaggtactggtggagtatttgatagtgtattaaccttatgtgtgacatgttctaatatagtcacattttcattatttttattataaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgcatattactggtgcaggaccattctttgatacagataaaggtttctctgaccattttcatgagt

SEQ ID NO: 3 - DNA, 20303 bp, Homo sapiens
ttcccatcaagccctagggctcctcgtggctgctgggagttgtagtctgaacgcttctatcttggcgagaagcgcctacgctccccctaccgagtcccgcggtaattcttaaagcacctgcaccgcccccccgccgcctgcagagggcgcagcaggtcttgcacctcttctgcatctcattctccaggcttcagacctgtctccctcattcaaaaaatatttattatcgagctcttacttgctacccagcactgatataggcactcaggaatacaacaatgaataagatagtagaaaaattctatatcctcataaggcttacgtttccatgtactgaaagcaatgaacaaataaatcttatcagagtgataagggttgtgaaggagattaaataagatggtgtgatataaagtatctgggagaaaacgttagggtgtgatattacggaaagccttcctaaaaaatgacattttaactgatgagaagaaaggatccagctgagagcaaacgcaaaagctttcttccttccacccttcatatttgacacaatgcaggattcctccaaaatgatttccaccaattctgccctcacagctctggcttgcagaattttccaccccaaaatgttagtatctacggcaccaggtcggcgagaatcctgactctgcaccctcctccccaactccatttcctttgcttcctccggcaggcggattacttgcccttacttgtcatggcgactgtccagctttgtgccaggagcctcgcaggggttgatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattgggtaagctcctgactgaacttgatgagtcctctctgagtcacgggctctcggctccgtgtattttcagctcgggaaaatcgctggggctgggggtggggcagtggggacttagcgagtttgggggtgagtgggatggaagcttggctagagggatcatcataggagttgcattgttgggagacctgggtgtagatgatggggatgttaggaccatccgaactcaaagttgaacgcctaggcagaggagtggagctttggggaaccttgagccggcctaaagcgtacttctttgcacatccacccggtgctgggcgtagggaatccctgaaataaaagatgcacaaagcattgaggtctgagacttttggatctcgaaacattgagaactcatagctgtatattttagagcccatggcatcctagtgaaaactggggctccattccgaaatgatcatttgggggtgatccggggagcccaagctgctaaggtcccacaacttccggacctttgtccttcctggagcgatctttccaggcagcccccggctccgctagatggagaaaatccaattgaaggctgtcagtcgtggaagtgagaagtgctaaaccaggggtttgcccgccaggccgaggaggaccgtcgcaatctgagaggcccggcagccctgttattgtttggctccacatttacatttctgcctcttgcagcagcatttccggtttctttttgccggagcagctcactattcacccgatgagaggggaggagagagagagaaaatgtcctttaggccggttcctcttacttggcagagggaggctgctattctccgcctgcatttctttttctggattacttagttatggcctttgcaaaggcaggggtatttgttttgatgcaaacctcaatccctccccttctttgaatggtgtgccccaccccccgggtcgcctgcaacctaggcggacgctaccatggcgtagacagggagggaaagaagtgtgcagaaggcaagcccggaggcactttcaagaatgagcatatctcatcttcccggagaaaaaaaaaaaagaatggtacgtctgagaatgaaattttgaaagagtgcaatgatgggtcgtttgataatttgtcgggaaaaacaatctacctgttatctagctttgggctaggccattccagttccagacgcaggctgaacgtcgtgaagcggaaggggcgggcccgcaggcgtccgtgtggtcctccgtgcagccctcggcccgagccggttcttcctggtaggaggcggaactcgaattcatttctcccgctgccccatctcttagctcgcggttgtttcattccgcagtttcttcccatgcacctgccgcgtaccggccactttgtgccgtacttacgtcatctttttcctaaatcgaggtggcatttacacacagcgccagtgcacacagcaagtgcacaggaagatgagttttggcccctaaccgctccgtgatgcctaccaagtcacagacccttttcatcgtcccagaaacgtttcatcacgtctcttcccagtcgattcccgaccccacctttattttgatctccataaccattttgcctgttggagaacttcatatagaatggaatcaggatgggcgctgtggctcacgcctgcactttggctcacgcctgcactttgggaggccgaggcgggcggattacttgaggataggagttccagaccagcgtggccaacgtggtgaatccccgtctctactaaaaaatacaaaaattagctgggcgtggtgggtgcctgtaatcccagctattcgggagggtgaggcaggagaatcgcttgaacccgggaggcagaggttgcagtgagccaagatcgtgccactacactccagcctgggcgacaagaacgaaactccgtctcaaaaaaaaggggggaatcatacattatgtgctcatttttgtcgggcttctgtccttcaatgtactgtctgacattcgttcatgttgtatatatcagtattttgctccttttcatttagtatagtccatcgattgtatatccgtccttttgatggccttttgagttgtttcccatttgcggttatgaaataaagctgctataaacattcttgtacaattctttttgtgatcatatgttttcgtgtttcttggagaaatacttaggaggggaattgtggaggaagtaaaaagtagctgtattttgaactttttcagaagctctgagttttccagagcggttgtaccattttacactccaactagcaaggtatgggagttattatggttgtgccacagccttccggacattaggtatgtcagtctttctaatgtggtatatccttgtggttgtaatttacagttctctattgactaaggatgttcagcattttttcatgtgcctattggccattcgtattttgtttgtaaagtagctcttcgagtcttttacctgttattttggtttttttgtttgtttttattgttcagttgtgggactgctttatactttctggatacaagtccttatcagatccatgagtcgtgaatgttttcttctgatctgttgcgggcctatttgtttgctttacagagtttacagaatcttaagaggagtggattaatcttttttatgttcagtatttgccttgtcctgtttaggacatctttttttttttttttaaccccagggtcatgaagatataatcttacattttcttttaggacctttatggtggtaagttttacagtaaggtccttaagccattaattaattcttaaaattaattgtttatggtgtgaggtgtaggagtcagtctctggtatctttcctgtatggaaatccagttattctgtctccacttgttgaaataggcttcctttctctactgaatgcttttaattttaattattttacagttggagtatagggctaccattttagtgctattttctttttttctttgttaatttttgagacagggactcacactgttgcccaggctagagtacaatggcacaatcaaggcttactgcagcctcgaacccctgggctcaagcagtcctctagcagcctcacgagtagctgggattactccaccacacccagctaactattttatttttttgtattgacaggatctcactatgttgcccaggctggtctcaaactgctggcctcaagctttcatcccatctcggcctcccaaagtgctgggattacaggtgtgagccaccatgcctgacctcttagtgctattttctatttatctcctctgttctctgctctctttaaacgttggaggaagaaacagtacccatcttacacaaactcttcagaaaacagaggaacagactgggcgcggtggctcatacctgtaatctcagcactttggtacgctgaggcaggggatcatttgaggtcgggagttcgagaccagcctggccaacacggcgaaaccccatctctactaaaatacaaaagtagctaggcgtgcaccatacctgtaatgccagttactcaggaggctgaggcacaagaatcccttgaacctgggaagcggaggttgcagtgagccgagattgcgccactgcactccagcctgggcaacagagtgagaccctgtctcagaaaaaaaaagaaagaaagaaaaaatagaggaatatttcccaacttgttttcgaagccaggataatcctggtaccaaaaccaaacaaggacattataagaaaagaaaatatagaccaatattcctgttagcatagacatgcaacagctaaccaattttagcaaaccaaacctggtaatatagaaaaaaggataaataggccagtcgcggtggctcacgcctgtaatcccagcactttgggaggctgaggcaggcagatcacttgaggtcaggagtttgagaccagcctgaccaacatggtgaaaccccgtttctaataaaaatacaaaaatcaggctgggcacggtggctcacgcctgtaatcccagcactttgggaggccgaggtgggcagatcacgaggtcaggagttcaagaccagcctgaccaatgtggtgaaacgccatctctactaaaaatacgaaaatcagccggtgtggtggcacctgcctgtaatcccagctactcaggaggctgaggcagaattgcttgaacccgggaggcagaggttgcagtgagccaagatcgtgccactgcactccagcctgggcgacagagcaagacttcatctcaaaaaaaaaaaaaattagctgggcatggtggtgggcacctgaaatcccagctactcgggagtctgaggcaggagaatcgcttgaacccaggaggcagaagttgcactgagctgggatcacaccattgcactccagcctgggcaacagagtgagactccatctcaaaaaaagaaaaagaaaaaggataaatacattctaaccaaataatgtttatctcatgattgtagctgattcaacattcaaaaattggcctggtgcagtagctcaggcctgtaatcccaacattttaggaggctgaggcaggaagatctcttgagcccaggatttcaagaccagcctgggcaacatagtcagactggtctttactggggggaaaaaaatcagtctgtgtaattcaccacattaacaaagggaaacataaaaaccctatgatcatttcaacagatgtagcaaaagcagttaatgatatcaacacatatgcatgattacaaaccaaccaacctcctagcaaactagggaaaggaaacttaactagtttgataacagggcgtccacagtcggagttccactagcagcatacataatggtagaaaactcagtgctgctgggggcggtggctcacgcctgtaatgccagcgctttgggaggcctaggcgggcggatcacgaggtcaggagatcgagactgtcctgactagcatgctgaaaccccgtctctactaaaaatacaaaaacaaaaaattagccgggcatggtggcgggcgcctatagtgccagctactcgggaggctgaggcgagagaatggcgtgaacccgggaggcggagcttgcagagcctagatcgtgccactgcactccagcctgggtgacagagtgagacttcgtctcaaaaaaaaaaaaaaaaaaaaaagaaaagaaaactcaacgctttttcctctaagatcaggaactagaaaaggatttgactctcacaacgttgataccatactggaggttttaaccaggcaagaaaaagaaataatgagggccgggtgcggtggctcaggcctgtaatcccagcactttgggaagccgagacgggtggatcacgaggtcaggagatcgagccatcctggctaacacggtgaaaccctgtctctactaaatatacaaaaaattagccgggcgtggtggcgggcgcctgtagtcccagctactcgggaggctgaggcaggagaatggcgtgaactcagggggcggagcttgcagtgagctgagatcgagccactgcactccagcctgggcgacagagcaagactgtgtctcaaaaaaaaaaaaagaaaaagaaataatgattagtggcccgatgtctcacgccagtaatcccagcactttgggaggccgaggtgggcagatcacctgaggtctggagttggagaccagcctgacaaagatggtgaaacctcgtctctattaaaatattaaaaaaatagccaggcgttggccgggtacagtggctcatgcctgtaaccccagcactttgggaggccgaggtgggtggatcacctgaggtcaggagttcaacaccagcctggccaacatggtgaaaccccatctctactaaaaatacaaaattagccgggcgtagtggcgggcgcctgtaatcccagctacttgggaggcttaggcaggagaatcgcttgaacctgggaggcggaggttgtagtgagccgagattgcaccattgcactccagcctgggtgacaaaagcaaaaactccgtctcaaaaaaaaaagaattagccaggggtagtggtgaacgcctgtagtcccagctactcaggaggcagaggcaggagaatcacttgaaccccggaggcagaggttgcagtgagccgagattgtcccattgcactccagcctaggcgagaagagcaaaattccatgtcaaaaaaaaaaaaaaaaaaggaaagaaaaaaaataacgattagaaaggaagaaatcaaacacattcacagccagtatgattctatacataccatggtcctaatggggccaggcgtggtggctcatgctgtaatcctagcacttttaggaggctgaggcaggtggcttccctgggaccagctggccaacatggtgaaaccccaactctaataaaaatacaaaaaatcagccaggcgtggtgagggcacctctaatcccagctactcaggaggctgaggcaggagaattgcttggacctgggaggcagaggttgcagtgagccgagatcgcgctattgcactccagcctgggcaacaagagtgaaactccggcagggtgtggtcttacgcctgtaatcccagcacttcgggaggctgagccaggccgatcacctgaggtcaggagtttgagaccaacctaacatggtgaaaccccgtctctactaaaaatacaagaattagctgggtgtagtggtgggcgcctgtaatcccagctacttgggaggctgagacagaagaattgcttgaacccaggaggtggaggttgcagtgagctgagatcatgccattgcacaccacgccgggcaacagagcgagattccgtctcaaaaaaaaaaaaaagatgaaactctatctcaaaaaaaaaaaaaagtcctaatggaaaatccataaaaagctaccaaaactaataaataaatatagcagggttgcaggttacagggcaatatagttatccctctatctgtaggggcttggttctgggactcctcacacaccaaacccacagatgtctaagtcccatatataagacggaatagtatttaacctacacatatcctcccatatagtttaaattatctagattacttacattacccccatacaatgaaaatgctaatgtacatgcaagtatgtatgtaagtacttgtactatattgtttagggaatcactggacagataggccttcaagactgataccagcagccactgttaagattctggtcaggcctgcccctgtttggggtctcagttgatctcattgccttcccacccagccaagggcacctgcatttctcttggctccctggccatttggaaggcctagttcagcctggcacatttgtatcctggcccactgatgctggtacccctgggaaggtcctgctctgaaaaacacggagattttagttgctactgaagatttgagagataaagacagggagacctgtctgtagacctgtgtccctccaagtgggattgagactttgggccccccatttcaggacagcacctcctggcctgttgactgaatagatccctgaaggaggtgtagttgcattttaggagtgggggtgggagcagtaccactgatccgcactaacaatcacacagttctctctagaataataatatagaacaagtgaaatagaacaattgcagaaagagctaacctttgttgagctcttactgtgtgcccagcactttcctcaactctacatttcccataatacatagagtactaggtaggcggggcttgggggctcacgcctgtaatcccagcactttaggaggccaaggggggtggatcacctgaggtcgggagttcaagaccagcctgactaacatggtgaaaccccgtctctactagaagtacaaaattagccaggtgtggtggcacatgcttgtagtcctagctactcagcaggctgaggcaggagaatcatttgaatccgggaggaggttgcagtaagcggagatagtgccactgtactccagcctgggcaataagagctgagactccgtctcaaaataaaataaaataaaataaaataaaataaaataaaataaaaaaagaaaagagcctgccattaaaggagctgtttggtaggggatgttttgtcagtgcaaacaacagaaaagtgggctgggcacagtggttcatgcctgtaatcccagcactttgggaggccaaggcgggcggatcacctgaagttgggagttcaagaccagcctgaccaatatggagaaaccccgtctctactaaaaatacaaaattagccgggcgcagtggccgatgcctgtaatcccagctactcgggaggctgaggcaggagaatcgcttgaacctgggaggcagaggttgcggtgagccgagatcgcaccattgcactccagcctggacgagagcaaaactctgtctcaaaaaaaaaaaaaaacagaaaagtgtaacaaacacttacagtaggcatgtttcttagcaaatctgatgacaaatttggcataaagaaagagagcatccctgaaaaaaaaaaaaagaaaaagaaagagagcatcctgcctgggcaacatagtgaaaccctgcctctacaaaaaaactcaaaaattggccgggtgcagtggctcacacctgtaatcccagcactttgggagtcggaggcgggaggatcacctgaggtcaggagttcgaaaccagcctggccaacatggcaaaaccccatctctactaaaaatacaaaaaattaatcaggcgcattggtgggcgcctgtaatcccagctactcaggaagttgaggcaagaggatcgcttgatactgggaggtggaggttacagtgagtcgagatcacaccactgcactctagcctgggtgacagggcgagactccgtctccaaaaaaaaaaagaaaaagaaaaagactaaaaaattagccaggcaggcctctgtggtcccagctacttgggaggctgaggcaggagaatcactgagcccaggagtggcaggctgtagtgagccatgattgcaccactgtaccctagcttgggcttcaaagcaagaccctgcctcaaaagaaaaaagaaagaaagaaagaacatggcgggccaggcacagtggctcacacctgtaatcccagcgctttgagaggccgaggcaggtggatcacaaggtcaggagttccacaccagcctggccaacatggtgaaaccctgtctctactaaaaatacaaaaaatcagcaggcagggtggtaggggcctgtaatcccagctactcgggaggctgaggcaggagaattgcttgaaaccagaaggcagaggttgcagtgagcctagactgcaccactgcactccagcctgggcgaaaagagccaaactccatctcaaaaaacaaacaaaaaaacaaaacaaaaaaaaacatggcagcctttgaaagcttgtctgggagaaggtgcgatgatggttgcataacttcgtgcaagatgctggtccacacaggggctgccccttgctctttctcgctctcttaacctctcatataacaggcttgtgtgttatgcacatttattgagcccaagcaggtgcaaggcattgtgatctaatactttggtcagcaagacaacaagatagatcactgccctgcccttaggaagtgtatatgctattagaggaaacagataaaataaacaaggaaaagtatcagacaatgtaagtgctatgagaatgcaaatgaggtgatgtgaattaaaataggatgacttaagtctgcacggaaggcccctacccccatgttcctggctagccaaggaaccaccagttgattagcagagaagggcagcccgtctagctagagcttttggggaagagggagtggttgttaagagatgagattaaagaagccgagacgggcccttcgtgagggggggttgtaatgcagggctgaggagtgtccgaagagaatgggcaggtgagcggtgagacagttgttcttccagaagctttgcagtgaaaggaatcaaagaaatggagccgtgtatcaggtggggaagggtgggggccaagggggtgtccttccccatacagagattgcaggctgagaatgactatatccttgttaacaggaggtgggagcagggcacggtagctcacacctgtaatcttggcactttaggaggcggaggcgggccgatcacctgaagtaaggagttcgagaccagcctggccaacatgcaaagccctgtctctactaaaaatacaaaaattagctgggtgtggtggtactcgcctgtaatcccagctactcgggagactgaggcaggagaatggcttgaacccggaaggtagaggttgcagtgagctgagatcatgccactgtgctccagcctaggtgacagagagagactccatctcaaaaaaaaaaaaaaatacaggaagggagttgggaatagggtgcacatttaggaagtcttggggatttagtggtgggaaggttggaagtccctctctgattgtcttttcctcaaagaagtgcatggctggtgtggggtggggcaggagtgcttgggttgtggtgaaacattggaagagagaatgtgaagcagccattcttttcctgctccacaggaagccgagctgtctcagacactggcatggtgttgggggagggggttccttctctgcaggcccaggtgacccagggttggaagtgtctcatgctggatccccacttttcctcttgcagcagccagactgccttccgggtcactgccatggaggagccgcagtcagatcctagcgtcgagccccctctgagtcaggaaacattttcagacctatggaaactgtgagtggatccattggaagggcaggccaccaccccgaccccaaccccagccccctagcagagacctgtgggaagcgaaaattcatgggactgactttctgctcttgtctttcagacttcctgaaaacaacgttctggtaaggacaagggttgggctggggacctggagggctggggggctggggggctgaggacctggtcctctgactgctcttttcacccatctacagtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacggtcagttgccctgaggggctggcttccatgagacttcaatgcctggccgtatccccctgcatttcttttgtttggaactttgggattcctcttcacccttaggcttcctgtcagtgtttttttatagtttacccacttaatgtgtgatctctgactcctgtcccaaagttgaatattccccccttgaatttgggcttttatccatcccatcacaccctcagcatctctcctggggatgcagaacttttctttttcttcatccacgtgtattccttggcttttgaaaataagctcctgaccaggcttggtggctcacacctgcaatcccagcactctcaaagaggccaaggcaggcagatcacctgagccccaggagttcaagaccagcctgggtaacatgatgaaacctcgtctctacaaaaaaatacaaaaaattagccaggcatggtggtgcacacctatagtcccagccactcaggaggctgaggtgggaagatcacttgaggccaggagatggaggctgcagtgagctgtgatcacaccactgtgctccagcctgagtgacagagcaagaccctatctcaaaaaaaaaaaaaaagaaaagctcctgaggtgtagacgccaactctctctagctcgctagtgggttgcaggaggtgcttacacatgtttgtttctttgctgccgtgttccagttgctttatctgttcacttgtgccctgactttcaactctgtctccttcctcttcctacagtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtgagcagctggggctggagagacgacagggctggttgcccagggtccccaggcctctgattcctcactgattgctcttaggtctggcccctcctcagcatcttatccgagtggaaggaaatttgcgtgtggagtatttggatgacagaaacacttttcgacatagtgtggtggtgccctatgagccgcctgaggtctggtttgcaactggggtctctgggaggaggggttaagggtggttgtcagtggccctccgggtgagcagtaggggggctttctcctgctgcttatttgacctccctataaccccatgagatgtgcaaagtaaatgggtttaactattgcacagttgaaaaaactgaagcttacgaggctaagggcctcccctgcttggctgggcgcagtggctcatgcctgtaatcccagcactttgggaggccaaggcaggcggatcacgaggttgggagatcgagaccatcctggctaacggtgaaaccccgtctctactgaaaaatacaaaaaaaaattagccgggcgtggtgctgggcacctgtagtcccagctactcgggaggctgaggaaggagaatggcgtgaacctgggcggtggagcttgcagtgagctgagatcacgccactgcactccagcctgggcgacagagcgagattccatctcaaaaaaaaaaaaaaaaggcctcccctgcttgccacaggtctccccaaggcgcactggcctcatcttgggcctgtgttatctcctaggttggctctgactgtaccaccatccactacaactacatgtgtaacagttcctgcatgggcggcatgaaccggaggcccatcctcaccatcatcacactggaagactccaggtcaggagccacttgccaccctgcacactggcctgctgtgccccagcctctgcttgccgctgacccctgggcccacctcttaccgatttcttccatactactacccatccacctctcatcacatttccggcgggaatctccttactgctcccactcagtttccttttctctggctttgggacctcttaacctgtggcttctcctcccacctcctggagctggagcttaggctccagaaaggacaagggtggttgggagtagatggagcctggttttttaaatgggacaggtaggacctgatttccttactgcctcttgcttctcttttcctatcctgagtagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgaggtaagcaagcaggacaagaagcggtggaggagaccaagggtgcagttatgcctcagattcacttttatcacctttccttgcctctttcctagcactgcccaacaacaccagctcctctccccagccaaagaagaaaccactggatggagaatatttcacccttcaggtactaagtcttgggacctcttatcaagtggaaagtttccagtctaacactcaaaatgccgttttcttcttgactgttttacctgcaattggggcatttgccatcagggggcagtgatgcctcaaagacaatggctcctggttgtagctaactaacttcagaacaccaacttataccataatatatattttaaaggaccagaccagctttcaaaaagaaaatagttaaagagagcatgaaaatggttctatgactttgcctgatacagatgctacttgacttacgatggagttacttctgataactcgtcgtaagttgaaatattgaaatattgtaagttgaaaatggatttaatacacctaatctaaggaacatcatagcttagcctagcctgctttttttttttttttttttttggagacagagtctcactctgctacccaggctggagtgcagtggcgggatctcggctcactgcaacctccgccttctgggttcaagcgattctcctgcctcagcccactgagtagctgggattacaggcacctgccccgacgcccagctaattttttgttatttatttcctttttttttagtagagatagaatttcaccatgttggccaggctagtctcgaactcctgaccttgtgatctgcctgccttggcctcccaaagtgctgggattacaggcgtgagccaccgcacctggcctgcctagcctacttttattttatttttaatggagacagcatcttgctctgttgcccaggctggattacagtgatgtgatcatagctcattataccctcctgggctcaagcaatccccctaactctgcctccccagtagctaggaccacaggcatacaccaccatacccagctaatttttaaaattttttgtagatagatagagtctcactatgttgcccaggctggtctctagcctacttttttgagacaaggtcttgctctgtcacccaggctggatagagtgcagtagtgcagtcacagctcactgcagcctccacctcccaggctccatccatcctcccagctcagcctcccaagttgcttcaactacaggcctgcaccaccatgcctggctaatttttatttatttatttttattttattttattttattttttgagactcagtctcactctgtcgccttaggctggagtgcagtggcatgatctcggctcactgctaacctctgcctcctgggtttcaagtgattctcctgcctcagcctcccgaatagctaggactacaagcgcctgctaccacgcccggctaatttgtgtatttttagtagagacagggtttcaccatgttggccaggctggtctcgaacttctgaccatgtgatcgccgcctcggcctcccaaagtgctgggattacaggtgtgagccaccacgcccggctaatttttatttatttatttaaagacagagtctcactctgtcactcaggctagagtgcagtggcaccatctcagctcactgcagccttgacctccctgggctccggtgatttcaccctcccaagtagctaggactacaggcacatgccacgacacccagctaattttttattttctgtgaagtcaaggtcttgctacgttgcccatgctggtatcaaacccctgggctcaatcaatccttccacctcagcctccccaagtattggggttacaggcatgagctaccacactcagccctagcctacttgaaacgtgttcagagcatttaagttaccctacagttgggcaaagtcatctaacacaaagccctttttatagtaataaaatgttgtatatctcatgtgatttattagatattgttactaaaagtgagaaacagcatggttgcatgaaaggaggcacagtcgaagccaggcacagcctgggcgcagagcgagactcaaaaaaagaaaaggccaggcgcactctcacgcctgtaatcccagcatttcgggaggctgaggcgggtggatcacctgaggtcaggagttcaagaccagcctagccaacatggtgaaaccccgtctctactaaaatacaaaaattaaccgggcgtgatggcaggtgcctgtaatcccagctacttgggaggctgaggcaggagaatcgcttgaaccaggaggcggaggttgcagggagccaagacggcgccactgcactccagcctgggcgatagagtgagactccgtctcagaaaaaaaagaaaagaaacgaggcacagtcgcatgcacatgtagtcccagttacttgagaggctaaggcaggaggatctcttgagcccaagagtttgagtccagcctgaacaacatagcaagacatcatctctaaaatttaaaaaagggccgggcacagtggctcacacctgtaatcccagcactttgggaggtggaggtgggtagatcacctgacgtcaggagttggaaaccagcctggctaacatggtgaagccccatctctactaaaaacacaaaaattagccagtgtgagacacgttgagtccacgtactcggaggctgaggcacaagaatcacttgaaccccagaggcggagattcgaatcagccaagattgcaccattgcactcccgcctgggcgacgagagtgagaccccatctcaaaataaataaataaatatttttaaaagtcagctgtataggtacttgaagtgcagtttctactaaatcgatgttgcttttgatccgtcataaagtcaaacaattgtaacttgaaccatcttttaactcaggtactgtgtatatacttacttctccccctcctctgttgctgcagatccgtgggcgtgagcgcttcgagatgttccgagagctgaatgaggccttggaactcaaggatgcccaggctgggaaggagccaggggggagcagggctcactccaggtgagtgacctcagccccttcctggccctactcccctgccttcctaggttggaaagccataggattccattctcatcctgccttcatggtcaaaggcagctgaccccatctcattgggtcccagccctgcacagacatttttttagtcttcctccggttgaatcctataaccacattcttgcctccacgtagtatccacagaacatccaaacccagggacgagtgtggatacttctttgccattctccgccaactccccagcccagagctggagggtctcaaggggcctaataattgtgtaatactgaatacagccagagtttcaggtcatatactcagccctgccatgcaccggcaggtcctaggtgacccccgtcaaactcagtttccttatatataaaatggggtaagggggccgggcgcagtggctcacgaatcccacactctgggaggccaaggcgagtggatcacctgaggtcgggagtttgagcccagcctgaccaacatggagaaaccccatctctactaaaaatacaaaagtagccgggcgtggtgatgcatgcctgtaatcccagctacctactcgggaggctgaggcaggagaatcgcttgaacccgggaggcagaggttgcggtgagctgagatctcaccattacactccagcctgggcaacaagagtgaaactccgtctcaaaaaagtataataaagtaaaatggggtaagggaagattacgagactaatacacactaatactctgaggtgctcagtaaacatatttgcatggggtgtggccaccatcttgatttgaattcccgttgtcccagccttaggcccttcaaagcattggtcagggaaaaggggcacagaccctctcactcatgtgatgtcatctctcctccctgcttctgtctcctacagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggtaggggcccacttcaccgtactaaccagggaagctgtccctcatgttgaattttctctaacttcaaggcccatatctgtgaaatgctggcatttgcacctacctcacagagtgcattgtgagggttaatgaaataatgtacatctggccttgaaaccaccttttattacatggggtctaaaacttgacccccttgagggtgcctgttccctctccctctccctgttggctggtgggttggtagtttctacagttgggcagctggttaggtagagggagttgtcaagtcttgctggcccagccaaaccctgtctgacaacctcttggtcgaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtggagctggaagggtcaacatcttttacattctgcaagcacatctgcattttcaccccacccttcccctccttctccctttttatatcccatttttatatcgatctcttattttacaataaaactttgctgccacctgtgtgtctgaggggtgaacgccagtgcaggctactggggtcagcaggtgcaggggtgagtgaggaggtgctgggaagcagccacctgagtctgcaatgagtgtggactggggggcccagtgcccgggttccgggaggggaacaaaggctggagactgggtcagtctgcgggctgcatgacaacaagggagggggtggctccattcataactcaggaaccaaccgtccctcctcccctccgcccacggctggcacaaggttctctgcctcccctgcttctaggattgggctgcttccccctcggcagcctctcaccaaggattacgggatttaaatgtcgtgatttacgaaggctgagcctccagggtggccatcttcgtccatcagaagtggcaggatacctgggttccaagggaacagggtgg

SEQ ID NO: 4 - DNA, 44505 bp, Homo sapiens
ttggtaatggaacagagaaatacaaatatagaaacgtttttatcctttagatcttctaatggtgcagatatttcttaaccaatttcaagagtgccttattcaaaaacaaaaaacctgcacatagaaaaagcaaacatgtttttttaaaaaaagccctgtaaaagtggttaatctgtcgtctgattttcaatgtatgactttaatcaattgcatattccttaattctgcaaatgacaattaaaattaggaatcagtacctgaaaacgccatttatacttttgagtttatattagaaacacggttttgatgaaatacctttttcggttcaatctttttgaattgtgaccacaaccaatagctacacttagattctacagaattagtaggaaagatacaaatgagaaggtctttctagacaatggatctcactgaacatcatcttagtgtgtagactttttccactgcagtggcccacatgtgctatggtgacagacggcagttggcattaccacttatatagtaaaatatctttaagacaaaaataaaagttatttaaagaatacgctggccctgaaacatgagctgtgccttaaactacatatcctacctccatgagtgaatgaataagtgggttttccgcctgcatctatgtgttaagacctattgtttgcttgtattaattgtagtcctgggataaaagcacagaggtaactttcactgctgctttttgtactctctccaatgttttggaggaaaaataagcacaaacaatagcctagagaaactgaatcgatcatacttgttgcagcttcgacaaacgtcaattttgctgcattagaatgggaaacatttttcagtctattgaaatgaattacaaacgtttttaaatagaaaattagttaaaaaattggaggctgcttaatcgatagctttctctataaacatacttggatttcacaaataagtaataccgtaaaaatcttcttctccaaagaaaaatccccacaaataaatctattgatacctagtgacaagtggaaccagataaaaatggaatctataagaattaacctaattgacagcgctctggagctaatccatttccattagttatttgttcacagtaggtactcctaaggacttgttgaattgcgggcttggcgcccgttctacggagagttcacagccttcgtgagtggggacagaaggcggctcggcccggtgattcaggtcgaaattcaagctgaacagcctgctgagaggtgggatccaccatccggacagtggggggctttgggggtgctgtgagactgggctgcgacccaggtccagcagggagtgtgcggcacagaccacaaggtcggcgaggccccctaacccgcgcccggccgggaacccgcagaccagcgacggggcagctgcggggccaggagcgccccaagacgggcgggcgctgaacccgagcccctgccgccgccctggccccgaacttccgccctcccaggacctgtcccggccgccccgagcggtactcgaaggccggggccgagatgccaccttccgcaggccgcgggaaaggcgcgccgagtcctgcagctgctctcccggttcgggaaacgcgcggggcgggggcgtcgggcttgggacaggggaggataccagggccaccttccccaacccaggccgcgggggcccggcctccccgatgcagaccacagcgccctcacgggctgccctcaggccgcgcagcgggcagccgccagccgtcaccccggggagcgtccgtggggtgcccaggcaccccaccccggcccggggcgctcagacggcagcagactgctgggcggcgcggggactactttccaccgccccctcgcgccccgccccttgtcctcgcgcggcggaacgctccgcgctgcgccggtggcggcaggatacagcggcttctgcgcgacttataagagctccttgtgcggcgccattttaagcctctcggtctgtggcagcagcgttggcccggccccgggagcggagagcgaggggaggcggagacggaggaaggtctgaggagcagcttcagtccccgccgagccgccaccgcaggtcgaggacggtcggactcccgcggcgggaggagcctgttcccctgaggtgcttgggcgctcctttccttatccttccggggctgctcccgcttcctctcggagccaaacttcgtagcaggcgcgcggtccgggcggcgggctgggcgcagccgggaggcctggggttgggagcggggagctcaggtgggggacggtgagggtgggccgcgcccggggcgcggagggcggcggccgggcccgggttccggtcgcgctgcctctctggggccctgggggcatcgcttgcggggagggggcgccgcgggggcgcgtacaggagcccggatggcaggcggggtgggggtgggggtgggggtctgtggtttccgtccggggctctggccttggccgagtttgggggagggacccggtgcctcgggatgcgccgggccctgggtggggggcggggtggggacggggggctccgccttctcagctcttgcggcgagttggggttcgggcgctgaggcagagacgccaccctaagtcccatcagtcctggggatcggaccagtggactttctcttaagatttcctctttcattcttaagaatagaagtgttattattttttttaatgccctggctatgtgagtttgaatcgaagcaactttaaaccttagagcaactaaactctaagtgcagcgggtgcgatgcgtcagtagggtgagcacataaaaaatccatgtcttgcacctgtattttagcgtactatgcaggtgagtgaaagcagtggataatgtactgggagtcttatggatttatggtagtgggtatgagaccctggtgaaataagggggtggaggaaggcgaaggtgatggcttactgtttcttaccaagtgaactgcaggattcagcctctgactcagaccgcttcgagaattttgttcgtagaaataatttaaatttattcaaatagtttgatggcagctaaaattgaattatagagcacgttttcttttcagcggagtgaatttttccttcgctccaaagctggccaaatggaattcaagcattgcaacttctttcagtgttttgtctggagagaggactttgaaccgagacttttcgaagttaagttcctatagcctgcttctgaatctgccaagcttgaaagctttggcagttgggtgtatgtagttgttgccttcgttctcttcccttttggagggagcgttgtctcctactttgtatcttccagacatctgtggtcttccccccacccctcgagtttgtgagtggtgaatgaagaaagactaggctgctggtatgcagaggtcggcaaaaggaaatcgaggagtggttttagtgaaatgagagctttgtatcatgaataatggtggcttaggctagacatcaacttgaagagacggcagcatttcctttcataaagtctaggctaatgtttttcagatcgctaagttgtagtttgtctggaatttaggaagccatttcagtatttgtcacttggtgaacgaacattcaataccttcagatgtcttcgtgttgacttgtattcatcctaagaaatagtaaatatagtctcaagtgttatttatgttatactgctggtttattctctgcttaaattattgacataaatttctactttggaggcttttcgtttgaactaaggctgtgcggaatttattttacttttatatttaaatctttgaaaaatctctgattaaaaaaaaagtacccttaaaggtttgaggatgtcctttcacaccagacaaaatttggttaatttgcgcccaatattcattactttgacctaacctttgttctgaaggccgtgtacaaggacaaggccctgagattattgcaacagtaacttgaaaaactttcagaagtctattctgtaggattaaaggaatgctgagactattcaagtttgaagtcctgggggtggggaaaaataaaaaacctgtgctagaaagcttagtatagcatgtaactttagagtcctgtggagtcctgagtctcccacagaccagaacagtcatttaaaagttttcaggaaaaaccaacttaaaaaaaaataaggtggctaattaaaaaaaaatgaagcatttaacagtgttcaggtttcagagtatggaagaggggttttttaaactgttatctgattatttcttttaccaacatgatatagaaaagtgtatttccagtattaaaatttatcagactgagcttactgttcctgttaatgactggaataaaaattggcataaatgagggtctgtatgcttgttttaataacaccaccaccaagatagaaaacgaggaggcaagtttctccaagggtattttgaaatgtgttagcaaaactattgcagatactcgtttttgttatagggtgaggtggggagaggcgcatgctaagtattgttgaaactagggatgtagagaattaaaagtttgaatataattattttgtagttataagtagcagtgaaattaaatctcctgcaatagactatagaagtatatttagccaaatgaaacttcagtgttattgaaatgaaataatacatctgtcctgttacaagattatttttatttctcttgtggtttcctagcttctgataatcaataattgtagatgagtaggtggtaagttttaagtttgtactttgagcttagtcggaagcatgcttgactgccaacccggggcacaaaggatgaaggcttttagaactggacaaacttctaacaaaaggtatttgcaactcttttgtagtgtgtcatgttgatttgtgacattgtttttgaaaatatgtgttaacttagttttcttgtagccctctttttattggaactgtggtatctattgttgaaactgcttgactgagaacatttttataccataaaagtaaatagtaaacatagcccaggagcggcttctggtttgtccatcgtatgtagccattgcctccttgtactctcattgagaagatactgatttgcagattcagttgtccttctctaacagactatttatgtaatattgcagttgtgattgtgataggtaagtggaccagtcggttaaaataaatactcaggtttcacaaaaggaaaataatatgatttgtgttgatctaaatgagtataggagttaactcctatagtttttcatcacttaaactcaggggaaagttctttatttcctctgtttacttaagaatgctgcttttgtgtttcatgcaagactgagcttgactcagtttgaaacctaggctcatctgttgaggcctgaaccctgctgtccttgaagtatgcatataatttgcttccttcctaaggaaaaataagctcttgaaagataaagtcaatcacattaggaacccatttttagggtttagccacttttttttttttttttttttaactcatgggcatctcttctgttaagagacattccccactctccaagtttccctcaagcctgaagcagcagagtgagtagtgttggagcatgttttcattgcatgcttgggtcatgttgagtgccctccagtggatatagtataatgcttgtgatttttttttttttaattccaaacaagtttatgtgggatatatttaggaatagttctgatgagggagaatcaactaagaaacctttgatttctaaaataattaatatcattactgctaattaaaatacaggcttgagaaaatgtcttctcagccaatatttgcagtagaaaagtcgggaggttttttaaggtcactttgagtaggcagttctgcttaaatatatcataatgataaaccagaatctcagtatagtactttaggaggtaaaagatcataatattcagttatattgatgaattacagcaactgaaattctcagaaaaaaattaatgaaaatgtgaattgtcaatttgtctaaaatcattcacagagtaaaacataagtgctcaacttgattatattaggaaatagatagaaataaaggtaattgagccagtgtatgtgacctaaaatataatgcccttagtgaccatagggttggtctcatttgtacatagtggtgggccatgatgaactgtgttttgccctttgaatttttccttaaaaagctttctctaggctcctatgttcatggtttttctgttagtaatattattttctgaaaatccatgtttcaaatcagaatctaattagcaacaggaatgaagcttattctaaattagtttttggaagttaaacggtcagcatatggaaatttttcagggtttagatttttaaaaatttgtttttcagaatatgttgctggaatgaaaacgttagcgtagggacggaaaatgacacttaccagtgattgctttactttgcctgtggaattcagtgtaattttgtggaaacattggtatatgattttttactacttaagaaatgtattgctatagttagggtttttttttttttaaaggcaagaatgcctcaagtgctttatgtgaatgattatttcaggatggattaaatattcctccatcaaggaccatacttgtaaatcagtgatttccaagttggtgcttagtatttacagcatttactgtctataagcttctgttctgatttttcaagagttttctgagaaatgagagtaggcttaaaagttctttgaaaaattatgtacatacaacttactgaaaaaaattgctaccggggacttaatttgtctcttgaaatgggctacttgccttcattaatgtagcatactacaatttgatgttcaagatatgttactaagaataagatcgctttcagaagccttatataggattggtcttactacattgtagtgggaatggctactcaaatgtctccagggccagttaggtattgggtaaatgggaccatgcagactattaaaaattgaagtgcacatgaagcagccagtcataagcagctccagccactgtgtgggaatatagtttatgttgccagatcatctgatttctttcccctaagtgggaaatccagatcaatgtacatctcttgatttgcaagtgttggtgaacaaaattcatattttaagatgctgtattcagcacaaattaaatacacttatttgctgaatactgccagtttgtccctctgcagtagtaccatttgaagtacagtgttttcataatgattctgtgaaatgactggttctgtgaatgtacataatttagcagataacattgttaaattattaggtttgtatttatttaggcacttgggaaatgccttgtgtcaattgattatagattaggagcttaaaagcaagatttatattatcaacttatttgtgaagactgggaaacccacatttttaaagttaggaattaagatggccaggttcaaggaaaagggggagaagtaactttcttattactcaaccatcttaaatagagttctttaagtgtatttttaagaggtctcaaaacttaatctgaagggacgtcaaatgctggacaaattctgtgtatacaactcaagtcagcccccaattttactggtctttaaatcatgtcctttttaccagaagtttgcatttctaagctaaactattactgttagactagatccaaaacttaaaaacagtttaggtaattaaaaattaattgaatataaacgttttacttaaattaatggcaaatggctttttggccaatttaagtttatgtaggcagttaaatcgattttggttaaatcttttgctgctaacaaggtatttccagattttgaaaagtggggtggcctggtgcctgtagtaccagcactttgggaggctggggagggtggatcacctgaggtcaggagttcgagactagcctggccgacgtggtgaatacaaaaattagccaggcatggtggcaggtgcctgtgatcccagctgcttggaagtctgaagcatgagaattgcttgaacctgggaagcggaggttgcagtgagctgagatcacgccactgcactccagctggggcaacagagcgagactccatctcaagaaagaaaagtggggtgtttagtcttcaaactccgtgtttaagtgactggagtgaaaatgtaaatcataggccggtgttggtttaaaaagcatcatctgaaaataatgctgtagtctgcaattatttttattacgatacgatggtgtaaaatacaagcagatcagtgaaccattcatgaaacattaatcctaaaggcgtctcaccccaagtctatcccacaatctccatgagacttcgtggaaccactgtaaagtttcttgtgtaatatcccagaagtttcctacctctggtatcttttgaacttgttgaaaaggcttttccaccccctctttatgatggtttgaagagtgtgaacatctgaatgatgctggggtgaaactgcttcataacacttccattttctcccctatttatttccatatttttattttttcactaatatccccacggttttacttctgttttagtaattcacatgttgctggactaattctttttaactgacttgtaacagatatgttaaaccgtttaaaacttggggggtatttttaacctactttaagttagttcaagttaatcagtctacatggcatataaaccttatgattaataaatcttaaatgctggtagctgagttggaagccaaagacgtacaaaaaagctgaagtgttaggtttagtgtgataagcttctcttactaacagggttttgtaatagcagaaatagatatatgcatatatatgtgcatatatatagcataccttattggatgtccatataaaaatgtgtaagaagttaaatttactgcaaaatttcttgggagtgcaatttgaagatgatcttaagtggtgatagtagtttgctacactgggggatagttgttgcaaactgctcctaattttcctttactgtgaagtaaactgaacagctgtaatagggattaggaactgtactccctctctctcttttttaagtataattaagtggttttggggtaagggtgtagggagtgagtgtctttgaagttttgcatatactagatgaatgccacatgtataagggaggaacaagggattcttggaaatatttttcaatccaagtaactttggaggcttccaagtggagttcattcccctgtgtaggaaagtgctggggtagacccttaaattcctttctgagccattgaaagaatgtcctcaaacttcgcttatactttatagttcatttagatacaaaagttacaaactgaatgctatttaggaaacgtaatacactgacataccgctctttaaatagattataaatttagtatatcaattttctggcattttgctgaattttattgtttagttttcaagcccaactatcttgttactttgtatatcgtagttgtcccccgttgatcactgtttcctgcttaattgtgctgtcgtttttcctgggtcctgattcagagtgtcagcattctgttccccatagaataagaagaggctagaaagtttacagatgagatatctaggaatgccagaagatcaggggtcaccgttgaggcagagtaattaattatggttaaaatggtgttgctgataagtgggtgctgggaaataattaaaatttgattttttagaagaatacttctcatgcttgaagagcgccctcattatatgctaaagggcctcaggtttttccttattgccattatgctgcagattctattacatttgtctgaaaagatctaagacagaagggctgtttaataccttcccttttctcctgaacttcccctctcctctcccccatcaggagctaagtaggaaccccttcaccttgttaccatcagatttcatcaatggtctgtctttacaatgaaggaagtagtactgcattctgggcagaggccagtcctgaggcatgccttttcaaggacattgttactttagttacactggctcttctgttttaactcttatcccccagactctaatcctgttgctttttttggtccccatctcccacctttcatcatctgaaatccattcattgtaacttctggaactcagtcgttagaaaatcctttatattctcaatcttgtgaatgttcctttctttcttattccagctgtaacctagccttctccccaagaatgctacttcccttgcagctctctcaagtggtgaatttttcccttcttgcacaccttataacactgaactaggaggtgtgtggactaaatgtctgcttttgttccttattgtcacttcttgacctttattttccaaaacttcaagctttgactttcatgtgatcaaattataccacccactgcctgtctttatttcaagcacctgcaaaccttcctgggtcattcacatccttctttgttcacttcattagctcttggctcattgtcactgtctcttatttctgtcataattcttggtgacatcagtatctatgtagagcaatactagtgaagatgtggtctggtaactgttacctgtatgaattaagataaggagttatgccagaatataagtcacctgtgtcactaagtttactgtttagcttactttttttgtagcaagattttgatgaaggacgcaatatgttgatttacagtctggtacaaattttgatgtagaagatgcttccaatatcctggtctcttagttccttgatttcttctccagtgatcttattttctaccctaactcaactacatattcccattgtcatatcctagaatattttgtcttttatctgtaactctgctctcttcccccaatctcatttcaagcatcccactttctaattcctctagtaaatacgtcagttccaacagcccatcaatcccattgggacctacagtttatctatccaagcttttccctgttcctcaccctcacttctatacagctgaagtttcatactgaattataatcactttctcgtatacacgtttaacaatcttgtccctccctggcttcatgcccagtgatctcttgtatctatgaccatgtcctttatcttctcctctgtcactggatgaactgtagccttccaagataaggccactcagttcatttgtacagcagattccatcccctcttgctctcaagaatattactgtggtatctctcttttcttgtctctactggctctttccatgagcaaacatggtattatcccattacaaaaaaaattttttctccgtctctccttccactcaccacctcagtctctgcttctctttcccgcaaaataaccttgaaaaattgctttatgtactcccgttttcttttgaacccctgccagtgaccaccacgttataaatttgtagttgtcatctcacttaatctgttagtagtatttggcaccattgctacagttgcttgaaatgccttttcattggtttccaggccaccatgtctgttagcagcttttcctcttacttcactagcatttccttctttgttttttctgttatctttctgacctctgttggagtggctgaaggtttagtccttgaatctttttttgttgtgcatatttactccagtatcatagctttatacagatggtatttacatctgtttgctaacgatttccaaattggtatccttaaactggtatccagctattttttggtcagcattttggatgtctaagaagcttctcaaactaaactgacctcccggttttccccaaagctgcatcttagtcttttccgaaatgcaattctgtctttccagttacctagcttaaaagcttgcagttcttgactcatctttctctcataccacgtatctgaattctctctgcaaaaaattgtctgttctcccttcagaataaagtcacgtgtcattttatgatggggatacattcagaaatgcgtcattaggagataatcatggttgtgtgaacatcagagtatacatagacaaacctagatggtatagcctactacacatctaggctatatggtgtggccaattactatgatgaatactgtaggtaattgtaacataaaggtaggtatttttatctaaacgtattgaaacatagaaaaagtacagtaaaaaatatggtatcaaaaataaaaaatggtacaactgtataaggcagttgtgatgaatggagcttgcaggatatgttgctctgggtgagtcagcgagtgacgattgagggaacgtgaaagatgtgggacatcactgtacactactgtagactttataaacactgtacacttgggctacactacatttttgtaaggttttaaaagacttttttctataataaaccttaaattactgtcacttttttactttatgaattcttaattttttaaacgttttcactcttgtaataacacgtagcttaaaacatacattgtacagctgtacaaaaattttctttatatctttataagcttttttatatttttaaaattactttttaccttttagcttttttgttgaaaaactaagacatgggccaggcgcggtggctcacgcctgtaatcccagcactttgggaggctgaggcaggcggatcacgaggtcaggagataagagaccatcctggctaacatggtgaaaccccgtctctactaaaaatacaaaaaattagccgggcgtggtggcgggcacctgtagtccgagctacttgggaggctgaggcaggagaatggcgtgaacccaggaggcggagtttgcagtgagccgagatagcgccactgcactccagtctgggcgacagagcggaaactccgtctcaaaaaaaaacaaacaaaaaactaagacatgaacacattagcctaggcctacagagggtcaggatcatcagtatcactgtatttccatctccacatcttgtccttctggaatgtcttcagaggcagtaaacataaatggagctgccacctcctgtgataacagtgccttctggaatacctcttgaaggacctacctgtggctgttttatagttaactttttttttttaagaagtaacagaaggagtacactctaatgataaaaagtatagtaagtacataaacctgtaacaatcattatcattatcaagtgtcatgtactggacataactgtatatgctatactttttttttttgagatggcatctcactctgtcacccaggctggagtgcagtggtgcgaggatagctcactgtaacctcagactcctgggctcaagtgatcctcctacctcagcctcccaagtagctgggactacaccaggcaccccaccatgcctggctaattaaaaaaaattttttgtagagacagggtctcactctgttgccagggctggccttgaattcctggcatcaagtaatcctcccactttggcctcacaaagtgcgaggattacaggtaagagccaccatgtctggcccactgtacttttatacaactgaagcacagtaaacctactgtggtttcgtttacaccagcatcaccacaaacaccatgagtagaacattgtgctgcgacgttaacgatggctacaacatcactaggtgataggaatttttcagctccattataatcttatgagaccactgttgtatgtgcagttcatcatccactgaaatgtccttatgtgatgcatgtcttcatatccaaaaatattaatcatttctcactgaagccatgccatgccatgccatcttttgcctgtattattatttttcagcttttattttagattcagggtgtacatgtgcaggtttgttagaaagagtatatcgtatgatgctgaagtttgggatacagttgaaccagtcacccaggtagtgagcatagtactcaatagataacgttctaacattactcctccttccctccctgttcttgtctctgtctattgtatctttatgtccatgtgtaccaaatgtttagctcattcttgtgagaacatgtggcatttgattttgtttctgtgttaatttgcttacaataaatagtctccagctgcatccacattgctacaaaggacatgattttgttcttttttataggctgcatcatattccatggtgtataggtaccacattttcttgatccagtctaccgttcatgggcatttgggttgattgtatctttgctattatggatggcttttgcctatattattggaaaggccttctaactggtgtccctgcttacaccgttttcccccttaaatgtgttttcaacatggtagccagagtaaccctttttataacaataaatcgtgtaacttttttgttcagaaacttacagggcttaccatttcattcagtaaaagctcaagctcctgtatagtcagaccatatccttcatcacctgttacttttctcctctgactcttcagcctttttgtttttcctcaaactgatgaagccttcatggctgatgtcagatgttttgcccattgagatcttccttgttgactcagttgcacttggtcatatgattttcatttatttggggtatctaatcataatctgaaagttggctacttatttttacccctttgagggtccttgccctgtttttgtatccctgatagcgggacagccagatatctggaacttacaggtgttcaataaagttttgttgaatgaatattctggaatcacccaaccttttttttcccctccacttatttttcttctccctttcacggcctgaaagatgtcctatgtatatggttccacttatcactctcatcccagtttgtgatatactattccattatattactattattaatacaattccattgaacttgctcttgctgacttcaccactggacctacatgttggccaaatggatactttataattttagtcttgacccctgcctttggcacatttcttacctctagcacagcactgtccagtaatccacactttctgagacagtggaaatgttcagtatctgtgctgttcagttggtagcaaccagctacccatgcctattaaacatttgaaatgtggctgtgtgactagtggcaattatgttggagagtacagttttagaaactcctgtttttcttacatggcactacatttagtatcacaatctaattgtgcaagccagataggtaggagtcatctttattcctgttatttaatttttctcatctactatatccagttcatcacatcaacagcgcctgttgtttctacctcctaaatatttctttagtctaactactacttgtccctagtgccaccaccatctatcagctggaatattgctatagctgccttacaggtttcccttctttcctgttctcttctagttttttgaattttagtcagcacgagattttaaaaactcaaataagattgtgttattcacctgcttaaaacctttcatgactttcagtgtcacgtagaacagaaaacacttttcttaccaaaggctagagagctctacgtgatctggctatttttaacgtttcattgcactcacccttttcctctataatcaaactactctgatctcaagggttagttcttgaaagatgatcatgttctttaatgactttaggtttttgtgtgttattttctatttctgggatgtttattctctgttccttacatgctggcccttttgcatccttcttcaggtctcagcttacatgttaccttcaagaagcctttgaccactctaagtgggcccttccttccacttctgctgtgtaatcccactcccttctcccacttgttaattagttacatacttttttgtaattgtttatttggttgctgtctccctctcaagaatgcagggaccatgtctgcattctgcagtaatcactactgcacacccagaatctattacagatcctggcatgtagctgatgcataaatatttgttgaatgaaagtctgtacattgtatttatgctattggtattgctatgacctgaaactaaaaggagttgtggaaaagatttcttatggaacagaaatatcccttttgattaatatcacaatctcgtaaattgagaaaacaaaaaaatatatactactggagcattcatgtatagttggagattatgactcatttattggtgtgtttttggactcagaacaaagatgagggaatattccttaaagctctgtattgaaataacgaaaagcagtcacattttaataatagaagcttcctagcttactctttctgtaatcttcttttcctaaatgtaagagagcctcataattatgaggcttattactagagtaaggctgtcaaaggcagcaaaatgtctttctgtttggaagaataacataaacttgacatgtatggtgggggacagaaggtttcaaaagtttaagaatctgtgttgtcttaacaaatagatgcttctcaaggagcttacgctagtggttactctgtccagtcagggttttttcttctttaacttgggttcatttcctgatggcacacatgaagtttggatcatatggtttgactttagctatggtccttagctatggggagcagcatcagcgacctgtgacatgtaaattaaaaatacaatgccagggcccttccccagcccctctgatagagaacctcttggccatctgtatttttagatgttccaggttagtctgattaacacccttggttaagaaccattgggaggatctgattgccagtttaaggggaccttcaagcctgtaggtctttatagttaaaaaaaaaaaaagattttaaaaatcatgcatatgttgtggctgaattctggtttagcacatactgcttttaatggcctgaaatgtttttcccaaataaattgtcttgttatagctttcatgtgtgatttggtccagcttcttgttttgaagatacttacgggggggaacactttgtgatttctcttagtaacatattaacccacttaaaaaccctttctattacaggtcttcacatttaggcttaatgtgcttaattcaaatgtaaaaatacacctgcctttgttctcagtgaaagtatgtaataaataaatgaggggttggcaaactactgcccaccatctgtttttttatggcctatgaactaagaatcgttttggatagctaaaaaaaaaaatcaaaaggataattattttgtgacgtgaaaattatatgaaattcaaatttcagtttctgtgaatgaagttttaatggaacacagccatccatgcttatgtaagtgtgcatattctctggctgttttcactgcaatagcagagttgagtagttgtgacaaagagtttatggcccacaaaacctaaaatatttactttctgatgctttacagaaaaagtttcctgaaccttattctagctatatgttgttcataaatgaatctttcgtggttctgaaggcatttaagaatctcttaggttataaattggctgggcgcagtggctcacgcctgtaatcccagcactttgggaggccgaggctggtggatcacgagggcaggagttcaagatcagcctagccaagatggtgaaaccctgtctccattaaaaaaaaaaaaaaaaaaaaaaaaatagctggggttggtggtgggcagtaatcccagctactcgggaggctgaggcagagaattgcttaaacccaggaggcggaggatgcagtgagccaagatcgcgccactgcactccagcctgggcaacagagtgaaacaccatctcaaaaaaaaaaaaaaaaaaaaaacactcttaggttataaataattgttgttagctctccaagcctccatattacattttgtgtgttctcctgttcacattttgagcattttattttttattagcacattcagttcatcaggtatttaagagcttaatatatgccaaagcatatattaagcgagaagctgtttctaaatgtactgtctcagccctcacagagttcacttcattaggctctttaaaatttctttctttaaaaggtcagcgtgctggtatagtggggaagggaaactcttacaacacgtcgagtagaggaaggttatcattatgggatataatttggaagtcattgagtacctgccattaattctgcctgtagtctgaatgtagagattaacatgtagaaacttttttgaaataaaatcttcaatttctttggcatatctagtactgtctagctaggcatatagtcaaagtatggtgtatatttcaagtattaaaagtttttttgggctgtagtcactgttgaaaggatatagttctttactattacatgtgatacctttatataaaattggctaacccctgtctttcatttatctgcaacactgactgttaccagttgtctctaactttggtatggggggtggaaatatgattagattgaaagggtacatgactgagccacaagcagacctggatttgaattttaactgaacggtttattagctattcttacattaatactgctaatcagttttcttgtgatatgaggaatgatgtcttctttatgaggttgctaggaagattcaatgagataacatactaggctcagaactgaagttgctaggaatttaattatgctaccttgttaaagtatgtcaaaggcagaattcagtgtttagctgataccacaaggcagtatcctaaaattatgctgtaaaagatataaagatgctgtaagtgactcagaaacctagtgactttgtaatgcagttgattcttagaatactgtcactttaacagaataggagctaggaatgaagaaatagttattaaattactaaaatagaaaatttattgacacatgtaaagtgacatttgcttaaatattgaaaaatttgtagtactatttccttgctttagaaaacattggttaccactttttttatttatagcagtttgtttttgccttgaggcaagatggttgactgagtagttgccacatttcttttgtacaaagtccatttcataggccatctagcttttatgcttagaaacatttccttaacgttatatttcagtatttggctaacctatatagggttaaattatataggctaacttctcggacagatatttctaataatttatgtatttggttctgcaaatgtatgcaaaaatatatgtacaaaggtatgcagatgccttgcatacttgatatatgttaaattttttttaatgtagacctttttcgttctctttaatgactatatggtattccaccatcccccgctcacctggacaactacagtaacctcctaaatggtgtttctactttgctattgccccttattgtcttttttcccctttatagctgctggagtgaattttagaaagcctaagtcatacatcacattgcttcatgggcatcccagtacactttggattttattttacatccttactgatctgattctcatctctgtctcttcatggttctctgccttctagttacactggtgacctttcaaaacctttaccacattgagttcattccttacttttcactctttctctgcctggagtgttctgccccatctttacgtggccagctgctcctcctctgatgaaatgtctcttcctcacaggccttccctgaccacccactagagtagcacatcttctacctcataaacttgtttattagtatttcttactctaaattttcttttaaattgcttaattccctaacagtagaatataagcttcactgtatgtatgatcttgttgactctcttactcattgttattgtaataccagtaacaaagggtgtttaaaatttgttcagtgggtgaatatatgttccatttaatggataaattattttttattcagtctcctgttgatggacatttgaataatttccatctttttctctatgaatgcctcacttggcatgcttctgacagtattgccacagaatacatttctgttataaaaattgaatttttaagtcaaagggtagttacactttaatggatagtggcagcttactatcaaaagtttctgctagtttcaccatatccttattagcagtagatattatcaatcttttcaatctttgccaatctgataagcaaaaagtaaatgggtttaaacatcctttgtatatattcattgctcactttatgtttttcctttgaaatgttatttcttgttctttccctgcagtatgattctttctttttttgacttgttcccagttttttgtgtactatggatattagcctttaattatgttacggatgttctagtatgttattttttgaattacttcaaatgtgatttgttgctcagattttaaaaactacatacacaaattatctcatgtttccctttttggtttcaatttcgactcatgcttaatcagttcatcgattgggcatggttttattcttaatatatacccgtattttatctcattttatttttttacgtgtaaatatttggtgaatataggtttaattttaatgtaaaataaggatgaaaaatgatagttggaattacaagcccatttctcctaatacttttaatcaagtaatccactaattgaaatattaccttcttcatttatgaaattgccacattatatctgggtgtttttctgcctactacagtctcttacccatttctttcctaataatacaatacttgaattgctgtggttgttgatttataatgttatcttaatgataacattataaatgtgatggaactggttcctccttatagttcttcttaaatcaagaacaagacatatcttcccatttactctcgtatgtatctcattttactgttatgaatgaaatctgtcctatttgtgtataggaaaatagtttttgtatgtaattgtgatatggccagttttattaaaaatttggttaaactaagagttgttttctgttcagccttatcatactataaaatccacataaaatgggtataaaagtgtcgcaggacactgggctcagatgattctcccacctcagcttcccaagtagctgggactacagcggcatatgccaccacacccagccaatttttaaataagttttaaaaatagtatttttagtagagacagggtttcaccatgttgcccaggctggtcttgaactcctggactcagacaatccacctgccttggcttcccaaagtgttgggattacaggtgtgagccaccacaccttgccgaattgcagccatatttaatacttttttccatcctattccctttgctgcccccaggcctcctgtattgatagcccgctattaagaagctagtgtatattctttgcatacttttacttcataaactatatgaagcattgttctgttttttaacttaattggtataaaattatattttggaaattcagtatattctgtgaaaattatttagaaaatgtgcctctgagataaagcctattcaggatgtatcttaaaggagatagctgtgctttaacattatcagtctttttggctgcttatgttaatataagttggagaaaaacagtctgctttttgtgataatatgttcttggagatggagtgaaagattgtttaaaaacattgtcttttttttcccctgaagtaccagtatttattttaggattatgttactgatcaaagatgctgtgtggagttactcattggtgagactaacaataaatcacacatgcaaaggatgttaccataatctaattattttaaacagtaaaattatattctaagacatccagttggcctatatgtgctatatcaatgactatcaaggggctttttatgtatactgtatacatgtacttcacaaaaatataaaaggatgacatcaaaaatctggcaagccaaaagcctacattacatgtagcaaataaataagcatatgaacttattggaatttaaaaccctgtaggatgggcgggtgatggtatgtatgttagatgtgtggacatatctattaaaagttgtgtcagataacagctggtgctgacaagcccttggtaagatggcagcatgttcaatatgttctgtgaaaattatctcagtttatgatctgtcagtattgtggagctatgcatgaaaggacttaaaattcttacccttaaactcagtaacagtgtttctagaacttctggtgatatgggaaattaagagaattatttatatgcaaaggtgtttattgcagcattgttggaataatagacaaaatggggaagaacaagctcagaatggaggaggtagcttatagtatagacatacgatacaatccagatgataatattttataatagtcttcacaaggaattttatatttttatttttaaaaatacatagcagtgagtttaatataccaaacataccaaaatgtcatcatttactgtgtggtggactcatatgatggagatgataaataaaaatattaatttatttgaggcatatatttatggctgaggaaggaagacagttatgaagaacagctcattctggaaacatactaatttttcccagccataaagagatttcctatttcttttttttttccatttaccttctgtttcctacctgagaagatttcatacttctaataaccatttgtgtacctatttaaagacagtaccaaaggcatacattttagtgtttggaggaccaagggtcatttgatgtttgatgcttattgactattcgaggatgacaagacaccttgagaacacacacacccacacccacacccacaccctcacccacccaccccacccccctccccgaagaaagctgtgaaggaagaaagcagaaaagaacctggagtgagttgtaacttaaaatgttagtgttgcatgaagtgtgttaaaacaggaagatttgaggaaattgcatacattttctagatggcaaagtattactggtgacagttaatgaaaatgcatatgcatgtgtttttagatttacaaattttactaagaactttttaaaaatccctgaaggtgtatcaaaagtttatcatgcttatgaaatagagtagcactttctaactttaaaacggggaataattctttggatcttgattattggaaaagtgaattatgaattgctagtataaaactgtggttttaaaatatgtctgctttatatttttatgtagcagatttactcctagttaataatactcaaacttactgaaaactaaggtaattaagataattctgtcctgatgggaagaggaaaaataacttcagtgtgaaatctattatatattagttgtggcaagatttctcccattgactttgactggagacatttatagggttaaaatcggaaatagcacggtgaattttgaagtatccttgtagttggaaagagtattatgttcatattgccaaaaaaaagatgcatggatgcattagactggatggaaaatacatgagaagttggctagccccctctttgtcaaaacatcacttggtggtgataaagctgttggaaaacacagcattctaatgtagtctgtagtttaatgataatctgtgtcttgaaacatttagcgtagtacttatacaaacctagatggcatagtgtactgcatgcctagcctatatagtatagcctgttgcttctagggtgtaaagctgtatagcgtgttactataggcagttgaaacagtggtatttatgtatccttttttttttttttaaattcttttaagagacagggtcttgctctgttgcccaggctggatgcattggtgtgatcatagctcactataaccttgaactcctaagtgatcctctttgcctcagcctccccagtggctaggactacaggcacatactaccacacctggctaatttttaacatttttttgtagagatggaatttcgctgtgttgtccaggctggtcttggaactcttgtgctgcagcaatccacccgcctcccaaagtgttagaattacaagccacttcgcctggcttgtttacctaaacatagaaaagatccagtaaaaatacagaattaaaatcttgtggggccactgtagcatatgtagtccatcttgactgaaatgtccttatgcagtgcatgattgtacttcataatttttaagcactcctccctcttgattggtacttagtggattttatcatttttgtttcttcataattctttctgaaatgtctactggttggacctttgatctcctgaattgatcgtgatttcttctgttgtattttttgtctttgtcatttttttgtactctaggcagttttctcaattttagtttctattcaactttttgtttttatttattctctccagtatttatggagatactaaattgaagtgttctgtttctctctccaccctatccctagtttcaagttttatctcagtttctatggagtcagttttttcgttgctttaaaaaaaaattttcctgaagtgattggtaagttttggctaattgggagcactagaattgggcccttaatggttggcagggtgtggtggaggagagacagcccttagtccaaaggctcaggccagaaaaagaaagaggaaggctttccttttcctttccggagcagggttctgccctaggtcttgcttggcagtctatttgatttctttagcagttaatgctcagttttttggcatatgtggatctgcctccagagcaggtacaaggtgagtgagtctatgctgttacctaattagatccccatttctaccctttgtttttacttctctatctactgataggtttttaccctccttcacctcatagggttgcagtgaagagcaagatgaatttttatttatgttgcataaattttaaaagctaaaaaatatatatgtaatgttgggaagtcccagtgtacaaatggctattgtaaatttggaacatgaacttgcttttttccattgtaaaaatgaaatcattataaattgcggtcaagttactaggtcagcccacacagagtttacccagtaatatgcgtaaatgttttgcctttgcatcaacaacaaggaaaaacagtactataaaaaaatgttcctggaagccggatgtatcaaagcacttctgaaatagctatatagcctatagacatgaccagttggtttctgagtctgttgacattggccaaaggagaagctcagtgtagaacatgtttggagtctccttttgcagaaatacattggaggctggagtggggaaccaatttttcagaaaggtggtgaagtagttacatagccactcttttaaagacagtcaaaagatagaaactaaggccaggtgttggctcacatctgagataggaaaatcacttgaacctgggaggcggaggttgcagtgagcccagtatgcacctctgcactccagcctggtttggcaagagaccaaaactctgtctcaaaaaaaaacaaaacatagttcacacttaaatattttattccatatctttacatacccaatatgttaatttatagttcaagatgaacttgtttgggacagattttgtaataaaggaaatcgtgttattagaaatatctagaggccatgagcccttaaactgttctaatttgcaagtagttccctgtgtgatgcagtttttttcaatattgcacaataaaggcaaaatacggacaaattagatgataagatttatataaatttttaaaatattgatcaaaatatgtatccatattggtaatatttgtatttataataaatcattgctgtaaatttgaacttagaaaaattttactaataaaggtgcttttgtgttgcaaactttcatttgaaaagtaatttttctttgtaccaaaaaatctaaaattcgctattctagtcaccaaaatttgctttatgaaaaataatttttgatggcactatatcagaaaacaacttgttaaagaaaatgtggagtttttaaaatcccactgtacctctgttatccaaaggggatctgtgaatttttctgtgaaaggttaaaaaaggagagacctttaggaattcagagagcagctgatttttgaatagtgttttcccctccctggcttttattattacaactctgtgctttttcatcaccatcctgaatatctataattaatatttatactattaataaaaagacatttttggtaaggaggagttttcactgaagttcagcagtgatggagctgtggttgaggtgtctggaggagaccatgaggtctgcgtttcactaacctggtaaaagaggatatgggttttttttgtgggtgtaatagtgacatttaacaggtatcccagtgacttaggagtattaatcaagctaaatttaaatcctaatgacttttgattaactttttttagggtatttgaagtataccatacaactgttttgaaaatccagcgtggacaatggctactcaaggtttgtgtcattaaatctttagttactgaattggggctctgcttcgttgccattaagccagtctggctgagatccccctgctttcctctctccctgcttacttgtcaggctaccttttgctccattttctgctcactcctcctaatggcttggtgaaatagcaaacaagccaccagcaggaatctagtctggatgactgcttctggagcctggatgcagtaccattcttccactgattcagtgagtaactgttaggtggttccctaagggattaggtatttcatcactgagctaaccctggctatcattctgcttttcttggctgtctttcagatttgactttatttctaaaaatatttcaatgggtcatatcacagattctttttttttaaattaaagtaacatttccaatctactaatgctaatactgtttcgtatttatagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctggtaagagtattatttttcattgccttactgaaagtcagaatgcagttttgagaactaaaaagttagtgtataatagtttaaataaaatgttgtggtgaagaaaagagagtaatagcaatgtcacttttaccatttaggatagcaaatacttaggtaaatgctgaactgtggatagtgagtgttgaattaaccttttccagatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtaagcaatgacatagctagctttttagtctgctttgaagtaaatgctcaaggggagtagtttcagaatgtctacccaataccagtacttgaaaactaacgatgtttctgaattcctgtattacaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttgggtaagaaaacatgtcagaatgcttgaagctaaaaagtagaagagtatactcacaatatttctgatgaggcttttttcttcttcccagttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaaggtaagagaattattctttatgtggttttcatggagcattggacacctccagtgtcatgtcattccatgcagtgttcctaacctttttggcaccagggaccagtttcgtggaaaacagtttttccatgaatgggttgtgggaatggtttctggatgacaccattccacctcagataatcaggcattagattctcatagggagcgtgcagcctagatccctcgcatgtgcagtccacactagggtttctactcctatgagactctcatggtgcagttgatctgacaggaggtagagctcaagccaggtaatgctcgctcacctgccacttacctcctgctgtgcagcccagttcatttctgttcttttaaatttttgagtttccatatgtaaagcactatgcgaagtagtagggatatggtaggcaagcttctcttcacacttttgttcttaggtgggatgtagatgttgggaataataacctaatatttaatttgtgtagtgggaagaagtggggctatgagggcacataacacaagttgaaactgactctttttgagggttcaaggagacctcttggaggaagtgatagttgagttcagtgttcaaggatgagaagggattcactaggtgaaggttaggtgagaaaacaacatctttgaaacgaaggaaggagatggaaagttttgggaatttaagaaatactaatagtaaggaggaagaaaggtttgaggtgaggctattgagatagacttagcagatctcatagggctttgtagagcatgtttaaaagcacaatgggaaatttcagcagaagcctgaaatgatgaaatttgtttttagaaaattggggcagtgttgaaagggaagatatacagggaatgaaaggacaagcatgaatgatcattttatggtatctgtttttaaggtggatataattaggaaaattaaagggccaaatgatgaggagttaagtgccagttctggttcaaattttcagtgaatcagttttgatataactttcatcttagggcattactcttgcctaccaacatagtttctaaatttttttcttttggtgtgatcactgtgggaagaaggaaattgggcccaaactgatacattgtttggaggactgggatgtctgaatttgagtggaatgctttaaaaggacaagttggatagggccccagtatgggggtctgagtgatggggtccaggaatacatttaggtccaatggcaagctggctgaaattcttgtataataaaataggttggtaatatggctcttctcagacatgtgatcaagattccttgactaacaagatatatatatatatctttctagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtaagtatatgtatctattctgagtcttgtgtatagcatctgcagttctaattagattacttttcttaggaaaaggtggtagaactttaactactgaaaataaatggtcctattcagtttgcagccaagatttacattcagagtacctgtcatctggattgtagctaaatatttaaggctagtttaggtagagttcttattatccatcaaaaatgatggcatatgttttgcttaataaaatttgtttgtaatttcagttttgagtaaacctaagatttgctaacagagctgtgaatttataggagaaaagacaaattctaatatagtacagttttatgtaaagtgattgctttattagtagatgctcatgagcagtttttgttttgttttaacttttaggttccgggtaatgtgcaggcttgttatataggtaaattgcatgtcacaggggtttcgtgtgcagattattttgtcacccaggcagtaagtattgtacccaataggtagtttttcagttctttacctcccacccgtaagtaggccccagtgtctgttgttcccttctttgtgcccgtgtgtactcagtgtttacctcccacttataagtgagaacatgtggtatttggttttctattcctatgttagtttgcttaggataatggcctccagctccatccatgttgctgaggaagacatcttggtatttttttatggctgcttagtattccatagtatatatgtaccacattttctttatctagtctaccattgatgggcatttaggttaattccatatctttgctattgtgaataatgctgcagtgaacatatgcatgcatgtgtctttatggtaaaaagatttctttttctttgggcatatacctaataataggattgctggattgaatggtaattctgtcaggttttttgagaaatcaccaaattgctttccacaatggctgaactaatttactttcccaccagcagtgtataagcattctcttttctcagcaacctcaccagcatctgtcattttttgactttttattagtagccattctaactggtgtgagacggtatctcattgtggttttgatttgcatttctctaatgatcagtgatgtcgagcttttcttcatatgtttcttggccacttgtatgtcttcttttgaaaagtgtctgttcatgtcctttgcccactttttaatggggttgttcttttttgcttgttaatttaagtttattgtaaactctggatattagacctttgtcagatgcatagtttgccagtactttctcccatgccagtactttctcccattctgtaggttgtctgtttactctgttgatttcttttgctgcgcagaagctctttatactgtcccatttgtcagtttttgtttttgttgcaacttctcttggcatcttcgtcatgaaatctttgccaggtcttatgtccagaatggtatttcctaggttatcttgcagagtttttacagttttaagttttatatttaagtctttaatccattctgagttgatttttgtacatcatgtaaggatggggtgcagtttcaatcttggatgtggctagccagttatcccagcaccatttattgaatagggagtcctttccccattgcttgtttttgtttacttgttaggtgtgcggcctaacttctgggctttcttttctgttccattggtctctgtgtctgtttgtataccagtaccatgctgtgattgtaaccttgtattaacagtatagcttgaagttgggtaaagtgattcctccagttttgttctttttgcttaggattgccttggctattcaggctcttttttgggttcatatgaatttttaaatagtttttttttaattatgtgaagaatgccattggtagtttggtaggaatagcattgaatctgtgaattgctttgggctgtatggccattttaacaatattgattcttcctgccatgaaatagaatgttttttcatttgttggtgtcatctctgatttctttgagcagtgttttttgtaattctcattgtagagatctttcacctccctggttagttgtattcctaggtattttattctttttgtggctttggtaaatgggattgcattcttgatttggcttgcagcttggatgttgttggtgtctagaaatgcttctgacttttgtacattgatttttatatcctgaaactttgctgaagtttattggatcaaggagcttttgggcagagattatggggttttctaggtatagaatcatattgtttgcaaacagacttcctatttggatgcattttctttctcttgcctgattatgagcagtgttttgccctgatattctgtattctcagtgaatagatgtcgtctaagtatgagaaacaatttttttctattctgagtatttttaagaaggcaacttatatgtggtactttgtatattgtgtatgttggcaattggggaaaagaatagatggtttgtactagggcctcttgggttctgtgtgtgtgtgtgtgtgtgtgtgtgtgtgtcatgaaaacagttactttttagctaccaagcattttttctcctttcagtaacccacctaacaacatttactcagaatttcaaagcaagcttcaaatcagtattgaaagaaggaaaaatataaaggcatttaatggaagaaaatgttgggaataaagtatagggctggcaacacttacttttctcacttattgagagtaattttacttgggaatttatgagagagaaagacattatgattgctccaggtaactactggcagaggaaccatagtcttggggatagacaaatgtggctgagttcatatagaatgaggggatgggatgtaaattctgtcagctgttccagcagtaacctgtaatgtaggctaaaaatacagattttgagatttatttaatcagaatccctggagtgttaatttttatatcaagatctcatagtgttttatttgaagtgacagggaggtctgtagatagctggacatgtatgggactggaagcttaggaatctttaagttcttccaggttattcttatgttcatttgtttattctgaaaatagcatctaatgtattttaagaaatggaataggcacatagtatacattgggtaacacaacagatagggtccccgtgcttaattcttagtcttgtgaaggtgacaaaaatacttaaaaatatgtgatcctaaattagaatgagtgttatgggagaaatgacagcaaatagtgatgagaattaatggggaggggaattgtctagatgagagggaaaaggtctccttgaaaaggggatgttaagtgggactgcaggatgagagggaaccgtctcttgtctatatgagaagtgagggttaaacgttttccaggtagagaaaaggaacaccatgtgctatgtcttagaaccagggatatccagtcttttggcttccctgggccacattggaagaagaataattgtcttgggctacacaccaaatacactaatgatagctgatgagctaaaacaaaaaaaaattgcaaaagaatctcataatgtttaagaaagtttacgaatttgtgttgggctacattcagagctgtcctaggccatgtggcccatgggctgcaggttggacaagcttgccttagaaggaaagagattggtcaggcacggtggctcacgcctgtaattccagcactttgggaggctgaggtgggcggatcatgaggtcaggagatcgagaccagcctggctaacacagtgaaaccccatctctactaaaaatacaaaaagttagccgggcgtggtggcaggcgcctgtagtcccagctacttgggaggctgaggcaggagaatggtgtgaacccgggaggcggagcttgcagtgagctgagatagcgccactgcacttcagcctgggcgacagagtgagactctatctcaaaaaaaaaaaaaagggaaagagattgtggagatccaggtgctgaagagaaggtctgcataaacagaacttagtaatgaggtggatggcctggtatgaggttgaggttaggtaagcagagccataacatgcaggactttctaggttcctataagatagttactactcatggagtttattcatgctttattccagctttggagccatagatacagaatactttggtcagtttggaaggctaggtgggatccaaattctaaacggttcctcagggttatactaaagtatttctattatcttaaaaggatgctgagacactttcgatggttgtttatcaatagcaaagcatcacagtggtgtgtttaaaatattaataatagcattgtatagattaacagtttgaatgaccaaaagctagaagaccagactactgagatgttacaggcttttaggaatgaaatagtttgcttttagaactcaatagcaaagggcagatgtctgagatgcctgaaagaatcatagaatgtaataatataggagctaagggagcaaccaaaaacggtttgtggaggggacaacattggtaccatgaagataaatggaaccctcagaaggcatccttaatttttgaacataataatttaagaagctgacttaaagtgacttaaaaggtcagtaggtagctggaaatgtatgatactagaatgcaagagaggcaggctagagatttggaagtttccctcttagtatataggggtaagggcagcagggaaggggaggtagaggtgccacagagtcatctgtatgggacttttttttttaccctagaactgctgaatcagaatgtgtgtgttttaaagtctctgtaggccattctgatggacatctggggttaaaatccattctcttagagttaatagttatgtaaagggagggaatgaagtcttaaagaggggaaagaaggtagtcatttcacaaatactgagcatcctgatcatcagtcttacgcagatcattctattagtagctggagctactatgaaaaaggaacccaacagaggtgatctttgtcttgtagggaaagtggagtaacttacactatgaaggagaagtgcagggtaccataagaattacagcagatagacctcatctgaggaaataaaacagacccgaaagatgaaggagacaaggaaaagtatctcttactgcattcagaagtgatttaagttgaagatggatgagcgaagttaatctactatgtgggcattgggcttccatttatactcctttgccagagtaaatgtcccccatttaagggtcctaaaggatggaagattgtaaaccttggaacacatgttttgtagtcagtgaattgtataaagtccctgacagtaagtgttttcatgccgtctttctggattgttcttaccccaggaatttacctagcttctttaggtctttagtcagatgtcaccttcacagtgaggtgacctaattatctatttaaaatcgcagccccactccattatttttctccatagccctttaatatcatctgacatactgtatggttttagtttattgtatatttttctgcctcttccaactagatcataaattctgagggtaggaacttctgaatatttttgttcactggtctatctgcagctcagaacaggacctggtactgaataaatatttttgaaatgattgaatggatgaaaagaaatgagtaataagaatattacctaagggggacagtggagataacaaaggctttttcggcttaggaaaggaacagtagctatttgagagtttgtcactagtgaggtgaactggcaaagtgaaggaaactgagcaacattctagaaaatgagaggaaatcaaatacttaggtgaaaggaagtaaactctggaaatacagaaggacacctcctaaggctagaaaagatatttaggattgataggcacttctagctaatgactagggccttatatcctttttaattttctaggtggaatgcaagctttaggacttcacctgacagatccaagtcaacgtcttgttcagaactgtctttggactctcaggaatctttcagatgctgcaactaaacaggtaaattctgagtaaactggtgccatgggaatagagtcaagatgagtatgtgcttgtactgaccatctgtttttatctccataggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggtaaattgtcaaagtagaatttacctttgttgcagaattgaaaatgaagcatctctagctgttggatggctgtctaagcatagtgatcaataagtaggaattgtattccttagtaagtaggaagtatggctgcgataggggtaagattctgaaatgtttgtgtagtcagaactacttttagttgataccaatagatttagtgtggtgggaattttagggtaagaaaatgattttgttgagttgtatgccagttcttccttctgtttttcaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggtaggtaaattcttacagtgatacctggctatctaaaaggaatgcataaatccaaaggatcctgaacttctttctttggtcattggttccccccatccgtcttcctgaagagctaatgacaaagtaaataaataaataattacacatttctatggctgcagagaaaataaggcatagtgtggccccagtgatatttccttggacacgtccttcacatggtcagtcttacaaaggttgggttaggtgtttcataaagtgttctcatttaatttacacaaaggcccacttccttaggaagaggtagagtcataatttgagatcaaatctgtgtaatttcagagcctcttacccttgcctcatcatgcattttgactataaatatttagcagtccgttttattatcttttctgtgagttaaacttttttcatggacctaagaatattcagaaataagtagtagcatttctgtactcttaaccacaaaaatctcaacctgaagctttgatacaaagtttgtgtcttaaaagtagcttcattaaaagtatagtctaatgacatttctgatttctcagactttaagaccttattaggttagtttagaaaacaaagatggagcctaccagaacagatgttaggaatctcattttgctggttgctttgtgtatgtactcatattggggctttggctttcttcatttattactgttggtattggcccatctccatgaggtgacttaatagaacgttgagggcaccttttattttaaatctcttttctaggaagaagagagtttttgtgtccttgtaagaatcaagttatttataaaagctgctaaatgtagcagaataataaccccttttaaaactcaaatccagaaacaggagaaacagatggtacttacatattgcaaaagctatcttccttctatacatgaggctgtcagctgaatagtcttggaagagtgaggagtgaatttttctgctggcaactcggttagttttagcagttggtgctaaaacttggcaaagttttcaccaaatacatggaagatatacaaaaatagagggggcatgtaaaagaaaaacgttgacatagtctgagcattactttctcatcttctctttttatataccttttacccagaatgattggtgcccttactgtaggaaagttgtctttgggattcagcgctgtatggaagctctgttgcactgtgtatgggggaggggtgctgctttgaattagtgctgccaggaggcctcttttcagtgacattcaagttaatggaatccttcttccttcctgaactaattgcaagttacggggaacttcgggtatataatgtaaataattacagtctaataattgttcctcaaactttacagaggagaatgccctgtttgttaaccatgtttcttttggcaggagggggtccgcatggaagaaatagttgaaggttgtaccggagcccttcacatcctagctcgggatgttcacaaccgaattgttatcagaggactaaataccattccattgtttgtgcaggtatgttttaagtgaagtgttctaggttttatgtccataaaatttccagattgtaatgactaataacatttcagaaaattagggaccataatagggttaccaacatttaattttatgaaaattccctacattttttggtcagtaagagaaacattgagacttgagaagagggaggagatttcacatttcacttttatgggtgcctagaggggagagctgacctgggctgccagaggcagggcatagacccccaaccaattctgggttttccaaatcttagatcagttagagctgcctctgaagaaagggtttatagctaaaaaatattatggaaatccagtgctccagagcattaaacaccccaagacataaaattcagagaatattatttactacagtgtgaatgcctcttgcactctgaattgggaatgtttgcaccacagtggggggcttgccatgttttagctttagatttaattaggttttgtttgtgttttctccttagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtgggtaagtaaaaaggaaccaaagcctttagcagatgtgtacattgaagtctcagtttttcctcaagggcctttttctccttgtctcttagcgacatatgcagctgctgttttgttccgaatgtctgaggacaagccacaagattacaagaaacggctttcagttgagctgaccagctctctcttcagaacagagccaatggcttggaatgaggtagggaaatgtgagcagttatttatctggtagtttcctagagcaggtatggcagcttgttctttcctctcaaaacacttagtacacattcatttgcattgatgtttccctggcttgagtatttcttctttatgctgtctagcaactgctctgaggaagaactataatacaagctttaaagagtctgttcagaatcattacaaataagttgtgttatttaaaattataattcataagggagaaagatgaaaaatgttaccagattaaagaagatttttcaaaaggatgtaaggaaagaggcagtgttaaacactgttaagaggacagtttatcagtattttttactaaactttaataaaacttttctatttgaatttctgctatgaatttttcttcagcatttgtcctcagtactacaggtggttccttgaaacattgtttctaataaaactagaacatcctgatattttatccattctatagagatcattgatggtacacagacatacagtggattatgtttgttgagtgaatggaaagagagattgttaggtttacaacgatgcagctcttgagaccggagtttaagatcagcctgggcaacatagtgaaaccccatctttagctgggcatggagatggatgcctatagtcctagctactggggagacgggggcaggaggattgcttgaacccaggagttaacagactgcactcagtgacagagccagactccaacacaaaaaaaaaaaaaaaaaaaaagcaaattaccagtgagtagtgtgttacttgggtttttaataggcatcttattaacatgttccaacttgagcccttaactttctccacctacccccttccacaaacctgttttcactgtcttctctgtcttagttaatgtcagctttgtctgtccagctgctcaggctaaaacttttctttcatataacacatcctatcagcagctcctgtttgtgggtaggcattttgcctttttttttttttttttttttaaactgctatatctctagcatgtagaacagtgcctggcagcacataataggtgcttaatataatatttgttgaaagaacaagtcagtgagtatttttaatgtgaggtgcaaagagaaaaaaaaatgtatctttgaggtgtggagttttgaagaacttccattttctaagcatttgtgtaatgttggagttacttgttccttttgtaatctgaaagtatgctttaaaaaaaattagtgtacttttgagaattttcattttgctttctattcttccttgctttgtgcatgtttatctagactgctgatcttggacttgatattggtgcccagggagaaccccttggatatcgccaggatggtatgtgtctcatatttctcgattaactccagatcaagctaaagttctaaaacttttatcagaagagccggtttgctcatctgggaaaccagtgttggcagaaaagtagtggcttcaattaaaagcagttcttaaattccagtcagcaacagtatctttaatggagcacagggaattcagagccacacaatgagtagcagtaggattacaccaccaacaaatacatgctactgctaggcctctgcagtgcaggatgttacaatttacctggctttttattctctttttggccagaggactcataatacctttgtctacaagctacccaaggaagataggaaaactcctgtttctaggctcagatctcgggtgggtttttacatagttgcattatcatcagggttttcttgaaaagctaatttaaatctgggtaatgaacatggaggatggcatagaccactaacaattataactgtcttacatttataaccgcatctgcttctacctaattatgaaaccactaaagcgcagattcttactgtgagaaataacatgtcaaccctaagataaaatatgttgaggtttcatggaaatagtgcctttccttagtacttttgtgggtgtcacttggcctttttgtcaagatagattacacctgccagacctcattattgtcttaatcctccttcccatgacttctcactgcctaggtggtcacacagtagattcctgcttcttctcctcgggaaccccaagtctcttgacaggggtaaatgcagagtgttcagggttagactaatgatgtgactaggccctgctggtgtgcctgtctgatggaaatagatgttatttgtgtagtctcatgggtggcctggcactgagtaattacttggctaaagaaagctggaggttgaagaggctagaaagcgttgttttctgacaagtttgctgctgaactttggatgccctaacctcagtgttaacgtctatgtctgcttctctcctctctcttttgccttccttcttgcctattttgttgacaccctgactcttctagatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaatcatcctttaggtaagaagttttaaaaagccagtttgggtaaaatacttttactctgcctacagaacttcagaaagacttggttggtagggtgggagtggtttaggctatttgtaaatctgccacaaaaacaggtatatactttgaaaggagatgtcttggaacattggaatgttctcagatttctggttgttatgtgatcatgtgtggaagttattaactttaatgttttttgccacagcttttgcaacttaatactcaaatgagtaacatttgctgttttaaacattaatagcagcctttctctctttatacagctgtattgtctgaacttgcattgtgattggcctgtagagttgctgagagggctcgaggggtgggctggtatctcagaaagtgcctgacacactaaccaagctgagtttcctatgggaacaattgaagtaaactttttgttctggtcctttttggtcgaggagtaacaatacaaatggattttgggagtgactcaagaagtgaagaatgcacaagaatggatcacaagatggaatttatcaaaccctagccttgcttgttaaatttttttttttttttttttaagaatatctgtaatggtactgactttgcttgctttgaagtagctctttttttttttttttttttttttttgcagtaactgttttttaagtctctcgtagtgttaagttatagtgaatactgctacagcaatttctaatttttaagaattgagtaatggtgtagaacactaattaattcataatcactctaattaattgtaatctgaataaagtgtaacaattgtgtagcctttttgtataaaatagacaaatagaaaatggtccaattagtttcctttttaatatgcttaaaataagcaggtggatctatttcatgtttttgatcaaaaactatttgggatatgtatgggtagggtaaatcagtaagaggtgttatttggaaccttgttttggacagtttaccagttgccttttatcccaaagttgttgtaacctgctgtgatacgatgcttcaagagaaaatgcggttataaaaaatggttcagaattaaacttttaattcattcgattgtgtcactctttctttttttcttgtcattaataaatggtttgtgagggtggtaagtaagtttatccaactcaactttatcctgaactttatcctgagatagatagatagatacatagtgaagtaatttgttgtagcgatagagagggccctactacttctctagatgcaatttgccaagtttctttagcatttggccctggattacgctggacccctaaaaaagtgtttgtttcagccttgcacattgtgattagttctgggctttggtgggagaaagccaagcagactatccatctatctatctctaccccaatccccctcagatgatttgattccattaaagtaaagtaatgaaataaatagtaactacccctacctggatttggatttgaatcaccttcacccaagtgtgactcattgggagaggggaattccttcaactggactttgaactaataataattaaataaattaaataggtttaagtgttgatgtttatctgaacataatcagaaccagaatgagccttccacagtacctgccagatgatcctatcaatcatcccggtggtaggtctgacaagcttagttcatctcatctcttcaggtagacctgtttgttaagacttgtgttttggtcttttaaaaagtaaaagaaagaagtgttttgtctttagggagagctctttctgatttactgtttttattttgatgatgattgatagtgactattaattatgagaaaaacatacattcagttctttttcgttaaaaatcagcttcatctggggaggcctttttgtgaatgcccttgctttgcaaaaatgaattcattgtggtctaaaatgtaattagggtttgaaaagaaacattcacatagctgatgatgtggtaggtctcaggttctgtctcatgtgtgataaaccaaaccagccagtatttcaagaagcctagggctattggtaggttaacaccgtgaagatcttcagtgaattcagcttttaacataaagagccaccgcacggtgtttcaattacagaaagaacaactttttaaaaccagagaatgttttcagagctttgctaattcattgtattttatttttgaagtttaaaaacttaacttaatgtgagagttaatccagttcaacaaggtggctctggaagtaccaagggtgaataatagccaagacgcttaggaaaaatagcacttgctctagcagctgtcaaggcctaactacttaatacaaagctaaagttagttaaccctgttacaggaatagggatataaatacagattaatacagaataatacacaggcatttaaagactcttcagagacagcgttggagatccgagtgggaggagagcaactttttcataaaggtgtgaaacagccgttggaataaaacaatacagaaaaacatccatgacccaacaaaatatcaagattttaaataaaggcagtagtactgatttttcttttgcctcaagcgccagtaaggcttggtagggcacaacgctgacagtctacactggacgcccatgtgtccaacaatctctgttcacac

SEQ ID NO: 5 - DNA, 780 bp, Homo sapiens
atggcttggtgaaatagcaaacaagccaccagcaggaatctagtctggatgactgcttctggagcctggatgcagtaccattcttccactgattcagtgagtaactgttaggtggttccctaagggattaggtatttcatcactgagctaaccctggctatcattctgcttttcttggctgtctttcagatttgactttatttctaaaaatatttcaatgggtcatatcacagattctttttttttaaattaaagtaacatttccaatctactaatgctaatactgtttcgtatttatagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctggtaagagtattatttttcattgccttactgaaagtcagaatgcagttttgagaactaaaaagttagtgtataatagtttaaataaaatgttgtggtgaagaaaagagagtaatagcaatgtcacttttaccatttaggatagcaaatacttaggtaaatgctgaactgtggatagtgagtgttgaattaaccttttccagatattgatggacagtatgcaatgactcgagctcagagggtacgagctgctat

SEQ ID NO: 6 - DNA, 2513 bp, Homo sapiens
cgcctcccggccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttgggattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtggaaaaaaaaaaaaaaaaaaaaaaaaaaaaaaaaaaaaaaaccc

SEQ ID NO: 7 - DNA, 3724 bp, Homo sapiens
tctccctcggcgccgccgccgccgcccgcggggctgggacccgatgcggttagagccgcggagcctggaagagccccgagcgtttctgctttgggacaaccatacatctaattccttaaagtagttttatatgtaaaacttgcaaagaatcagaacaatgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaaagataactgagaaaatgaaagctcactctggattccacactgcactgttaataactctcagcaggcaaagaccgattgcataggaattgcacaatccatgaacagcattagaatttacagcaagaacagaaataaaatactatataatttaaataatgtaaacgcaaacagggtttgatagcacttaaactagttcatttcaaaattaagctttagaataatgcgcaatttcatgttatgccttaagtccaaaaaggtaaactttgaagattgtttgtatctttttttaaaaaacaaaacaaaacaaaaatccccaaaatatatagaaatgatggagaaggaaaaaaaaaaaaaaaaa

SEQ ID NO: 8 - DNA, 845 bp, Homo sapiens
caagatcctcatcaggaggaaaagtaaattgttcactaccatcctctagtatcctaatctggtcttgttgttggctaacttcagcagttactattctgtgactggtgtaatattaaccaaataaattactggatttgttctacaaatattatgtcttagattggttctttcctgtctctgaaaataaagtcttgcaatgaaaataaattattttacaacagttaattagcaatgtaaaatttattgaaaatgtatttgctttttctgtaaatcatctgtgaatccagaggggaaaaatatgacaaagaaagctatataagatattattttattttacagagtaacagactagctagagacaatgaattaagggaaaatgacaaagaacagctcaaagcaatttctacacgagatcctctctctgaaatcactgagcaggagaaagattttctatggagtcacaggtaagtgctaaaatggagattctctgtttctttttctttattacagaaaaaataactgaatttggctgatctcagcatgtttttaccatacctattggaataaataaagcagaatttacatgatttttaaactataaacattgcctttttaaaaacaatggttgtaaattgatatttgtggaaaatcatactacattggtagttggcacattaaatgctttttcttactctgaattcctgatatgactttctttaggattgtttaaaatattctagtagttttaggtcaatttagatgtgatttagttggtctagatattataatttttaggggttccctttcatttttcttttttcttacgtttcttcaaatagtataat

SEQ ID NO: 9 - DNA, 497 bp, Homo sapiens
gaaagcctctctaattttgtgacatttgagcaaagacctgaaggtattaacatcatttgctccaaactgaccaaactgttcttattacttataggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaaagataactgagaaaatgaaagctcactctggattccacactgcactgttaataactctcagcaggcaaagaccgattgcataggaattgcacaatccatgaacagcattagaatttacagcaagaacaga

SEQ ID NO: 10 - DNA, 294 bp, Homo sapiens
  misc_feature (246)..(253): n is a, c, g, or t
ccagtggtatagaaatcttcgatttttaaattcttaattttaggttgcagtttcatcactgtctgcggtaatcaagtttttagaactcttatcagatgattccaactttggacagtttgaactgactacttttgacttcagccagtatatgaaattggatattgcagcagtcagagcccttaacctttttcaggtaaaaaaaaaaaaaaaaaaaaaaaaaagggttaaaaatgttgaatggttaannnnnnnngacagatagtgaagaaggcttagaaaggagctaaaagagtt

SEQ ID NO: 11 - DNA, 994 bp, Homo sapiens
ggacaaagcagtaaaaccgaacatatgtcttcaagcagtgagaatacgtccacaccttcatctaatgccaagaggcagaatcagctccatccaagttctgcacagagtagaagtggtcagcctcaaaaggctgccacttgcaaagtttcttctattaaccaagaaacaatacagacttattgtgtagaagatactccaatatgtttttcaagatgtagttcattatcatctttgtcatcagctgaagatgaaataggatgtaatcagacgacacaggaagcagattctgctaataccctgcaaatagcagaaataaaagaaaagattggaactaggtcagctgaagatcctgtgagcgaagttccagcagtgtcacagcaccctagaaccaaatccagcagactgcagggttctagtttatcttcagaatcagccaggcacaaagctgttgaattttcttcaggagcgaaatctccctccaaaagtggtgctcagacacccaaaagtccacctgaacactatgttcaggagaccccactcatgtttagcagatgtacttctgtcagttcacttgatagttttgagagtcgttcgattgccagctccgttcagagtgaaccatgcagtggaatggtaagtggcattataagccccagtgatcttccagatagccctggacaaaccatgccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacggaaagtactccagatggattttcttgttcatccagcctgagtgctctgagcctcgatgagccatttatacagaaagatgtggaattaagaataatgcctccagttcaggaaaatgacaatg

SEQ ID NO: 12 - DNA, 284 bp, Homo sapiens
gtactggtggagtatttgatagtgtattaaccttatgtgtgacatgttctaatatagtcacattttcattatttttattataaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgcatattactggtgcaggaccattctttgatacagataaaggtttctctgaccattttc

SEQ ID NO: 13 - DNA, 1648 bp, Homo sapiens
gccctgactttcaactctgtctccttcctcttcctacagtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtgagcagctggggctggagagacgacagggctggttgcccagggtccccaggcctctgattcctcactgattgctcttaggtctggcccctcctcagcatcttatccgagtggaaggaaatttgcgtgtggagtatttggatgacagaaacacttttcgacatagtgtggtggtgccctatgagccgcctgaggtctggtttgcaactggggtctctgggaggaggggttaagggtggttgtcagtggccctccgggtgagcagtaggggggctttctcctgctgcttatttgacctccctataaccccatgagatgtgcaaagtaaatgggtttaactattgcacagttgaaaaaactgaagcttacgaggctaagggcctcccctgcttggctgggcgcagtggctcatgcctgtaatcccagcactttgggaggccaaggcaggcggatcacgaggttgggagatcgagaccatcctggctaacggtgaaaccccgtctctactgaaaaatacaaaaaaaaattagccgggcgtggtgctgggcacctgtagtcccagctactcgggaggctgaggaaggagaatggcgtgaacctgggcggtggagcttgcagtgagctgagatcacgccactgcactccagcctgggcgacagagcgagattccatctcaaaaaaaaaaaaaaaaggcctcccctgcttgccacaggtctccccaaggcgcactggcctcatcttgggcctgtgttatctcctaggttggctctgactgtaccaccatccactacaactacatgtgtaacagttcctgcatgggcggcatgaaccggaggcccatcctcaccatcatcacactggaagactccaggtcaggagccacttgccaccctgcacactggcctgctgtgccccagcctctgcttgccgctgacccctgggcccacctcttaccgatttcttccatactactacccatccacctctcatcacatttccggcgggaatctccttactgctcccactcagtttccttttctctggctttgggacctcttaacctgtggcttctcctcccacctcctggagctggagcttaggctccagaaaggacaagggtggttgggagtagatggagcctggttttttaaatgggacaggtaggacctgatttccttactgcctcttgcttctcttttcctatcctgagtagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgaggtaagcaagcaggacaagaagcggtggaggagaccaagggtgcagttatgcctcagattcacttttatcaccttt

SEQ ID NO: 14 - DNA, 623 bp, Homo sapiens
taagacatatatttttgtttgaaatacactgaaactggtttcaaaatattcgttttaagggtaaagaaaaaagttaaaaaatctatttacataaaaaataagaacactgatttttgtgaatactgggaactatgaaaatactatagttgagaccttcaatgactttctagtaactcagcagcatctcagggccaaaaatttaatcagtggaaaaatagcctcaattcttaccatccacaaaatggatccagacaactgttcaaactgatgggacccactccatcgagatttcactgtagctagaccaaaatcacctatttttactgtgaggtcttcatgaagaaatatatctgaggtgtagtaagtaaaggaaaacagtagatctcattttcctatcagagcaagcattatgaagagtttaggtaagagatctaatttctataattctgtaatataatattctttaaaacatagtacttcatctttcctcttagagtcaataagtatgtctaaaacaatgattagttctatttagcctatataacctgcttttaagatttttggggcttgaaatgtgttaggatgaggtgagatgctttcctaagtttataggagaacctaaa

(19) United States
(12) Patent Application Publication
(10) Pub. No.: US 2008/0145852 A1
(43) Pub. Date: Jun. 19, 2008

(54) Methods and compositions for detecting adenoma

(76) Inventor: Anthony P. Shuber, Mendon, MA (US)

Correspondence Address: GOODWIN PROCTER LLP;PATENT ADMINISTRATOR, EXCHANGE PLACE, BOSTON, MA, 02109-2881, US

(21) Appl. No.: 11/897,981

(22) Filed: Aug. 31, 2007

Related U.S. Application Data
(63) Continuation of application No. PCT/US2006/007493, filed on Mar. 1, 2006.
(60) Provisional application No. 60/657,841, filed on Mar. 1, 2005.

(51) Int. Cl. C12Q 1/68

(52) U.S. Cl. 435/6

(57) ABSTRACT

Aspects of the invention relate to methods and compositions for detecting adenomas in biological samples. Aspects of the invention provide panels of genetic markers that can be used to detect adenomas in biological samples with different levels of sensitivity. Embodiments of the invention may be used to screen stool samples for one or more indicia of colorectal adenoma(s).

### RELATED APPLICATIONS

This application claims priority under 35 USC § 119(e) to U.S. Provisional Application Ser. No. 60/657,841 filed Mar. 1, 2005, and entitled “Methods and Compositions for Detecting Adenoma.”

### FIELD OF THE INVENTION

The invention relates to methods and compositions for detecting indicia of cancer in a biological sample.

### BACKGROUND OF THE INVENTION

Methods for detecting indicia of cancer based on the detection of certain genetic abnormalities in biological samples are known. However, there remains a need in the art for improved methods for detecting certain forms of cancer and for screening patient samples to identify those with indicia of cancer.

### SUMMARY OF THE INVENTION

The invention provides methods and compositions for detecting early signs of cancer or precancer by detecting adenomas. In particular, aspects of the invention are useful for detecting indicia of precancer or early stage cancer (e.g., of the colon) in a subject by detecting one or more genetic abnormalities indicative of an adenoma. Aspects of the invention include interrogating a biological sample (e.g., a stool sample) for the presence of one or more markers that are informative of adenoma. Panels or groups of informative markers may be used as sensitive detection assays to screen patient samples for the presence of adenoma. Aspects of the invention include detecting the presence of an adenoma with a sensitivity of greater than about 60%, greater than about 70%, greater than about 80%, greater than about 90%, or greater than about 95%. A sensitivity of 95% means that 95% of adenomas are detected and 5% of adenomas are not detected. A 95% sensitivity results in a 5% false negatives. Accordingly, aspects of the invention include adenoma detection assays with a false negative percentage of less than about 40%, less than about 30%, less than about 20%, less than about 10%, or less than about 5%. Accordingly, certain embodiments of the invention include diagnosing the presence or absence of an adenoma in a patient with high level of confidence.

Aspects of the invention include detecting adenoma by interrogating a biological sample for the presence of one or more markers belonging to an informative panel of markers. An informative panel is a panel that contains an informative combination of genetic markers. The biological sample may be a tissue biopsy sample. Alternatively, the biological sample may be a fluid, mucus, solid or other biological product that can contain biological material such as cells and cellular debris. In one embodiment, a colonic adenoma may be detected by interrogating a stool sample. In another embodiment, a colonic adenoma may be detected by interrogating a colon biopsy (e.g., a polyp or other tissue biopsy from the colon). Similarly, adenomas in other tissues (e.g., other gastro-intestinal tissues, pituitary tissue, lung, kidney, liver, or other epithelial, secretory, or glandular tissue, etc.) may be detected by interrogating appropriate biological samples from those tissues.

Aspects of the invention include panels of markers that are informative for adenoma. An informative panel may include a plurality of single base mutations (substitutions, insertions, or deletions) that are associated with adenoma. An informative panel may include one or more regions that contain a cluster of mutations that are associated with adenoma. An informative panel may include one or more CpG regions that are hypermethylated in adenomatous tissue. An informative panel of markers may include a combination of two or more of the above. Embodiments of the invention include panels of markers that are greater than about 60% informative, greater than about 70% informative, greater than about 80% informative, greater than about 90% informative, or greater than about 95% informative.

Accordingly, aspects of the invention include screening patient samples with panels of markers of different levels of sensitivity. In certain embodiments, a patient sample may be screened using a marker panel that is small and not as informative as a larger panel containing more markers. However, the advantage of using a smaller marker panel may be a reduced cost. Reduced cost may be desirable when an assay is being offered to a larger number of patients, particularly when the patients have no signs or risk factors for cancer. For example, a panel of relatively low informativeness (e.g. about 60%) may be used when screening a large number of young individuals who have no risk factors for cancer (e.g., no family or individual history of adenoma, colon cancer, polyps, or any other tumor/cancer etc.). In contrast, if a patient has a polyp, genetic risk factors, or other indicia of cancer or precancer, it may be more appropriate to use a panel of markers of higher informativeness (e.g., about 80% or higher). For example, biopsy samples taken from a patient (e.g., taken from the colon during a colonoscopy) suspected of being cancerous or precancerous based on physical examination may be interrogated with a highly informative panel.

Aspects of the invention also may include screening subjects repeatedly for the presence of one or more markers of adenoma. For example, a panel of markers may be used as a regular assay for indicia of cancer (e.g., colon cancer). Such an assay may be part of a routine medical exam. For example, an assay (e.g., using one or more of the marker panels disclosed herein) for one or more indicia of adenoma may be performed approximately every six months, approximately once a year, approximately once every two or more years (e.g., every 3, 4, 5, 6, 7, 8, 9, or 10 years). In some embodiments, an assay may be performed as part of a general medical screen or checkup. It should be appreciated that a panel of markers that has a relatively low informativeness may actually become more informative when used repeatedly to test individuals in a population. As adenomas grow and develop, additional markers may appear. Accordingly, an adenoma that was not detected in a first assay may be detected in a subsequent assay if certain markers of the panel that were not initially present in the adenoma subsequently appear as the adenoma develops. In certain embodiments, regular screening may be initiated in individuals that are 40 years old or older (e.g., 50, 60, 70, or older). In some embodiments, the frequency of the assay may be increased for older individuals.

In another aspect, the invention provides groups of nucleic acid probes or primers that are useful for interrogating a biological sample for the presence of each genetic abnormality included in a genetic abnormality panel that is at least 60% informative for adenoma. The nucleic acid primers or probes (e.g., oligonucleotides) may be provided in a kit. The kit may include instructions for interrogating a biological sample for a plurality of genetic markers belonging to an informative genetic panel (e.g., a genetic panel that is at least 60% informative for adenoma).

### DESCRIPTION OF SEQUENCES

SEQ ID NO:1—Human APC sequence (GenBank reference NM_{—}000038).
SEQ ID NO:2—Human Kras sequence (GenBank reference AF285779).
SEQ ID NO:3—Human P53 sequence (GenBank reference U94788).
SEQ ID NO:4—Human B-catenin sequence (GenBank reference AY463360).
SEQ ID NO:5—DNA sequence encompassing human B-catenin codons 20-51.
SEQ ID NO:6—Human Braf sequence (GenBank reference M95712).
SEQ ID NO:7—Human PIK3CA sequence (GenBank reference NM_{—}006218).
SEQ ID NO:8—DNA sequence of exon 9 of human PIK3CA.
SEQ ID NO:9—DNA sequence of exon 20 of human PIK3CA.
SEQ ID NO:10—Human genomic Bat-26 sequence.
SEQ ID NO:11—DNA sequence encompassing human APC codons 1286-1513.
SEQ ID NO:12—DNA sequence of a portion of human Kras Cp2 (HUMRASK02 exon 1).
SEQ ID NO:13—DNA sequence of a portion of human P53 (HSP53).
SEQ ID NO:14—DNA sequence of exon 15 of human Braf.

### DETAILED DESCRIPTION OF THE INVENTION

The invention provides methods and compositions for detecting the presence of one or more adenomas in a biological sample. According to aspects of the invention, genetic assays involving a combination of different molecular markers that are informative for adenoma can be used to detect adenomas with a high level of confidence.

It is difficult to develop sensitive and cost-effective cancer screens that can detect early stages of cancer in a population with a high level of confidence. Although mutations associated with cancer are known and methods for interrogating biological samples for the presence of one or more mutations are known, it is not economically realistic to screen subjects for all known genetic abnormalities. However, if subjects are screened only for a handful of genetic abnormalities, many cancerous or precancerous lesions may not be detected. Accordingly, sensitive and cost-effective cancer screens require selecting those markers that are sufficiently informative for appropriate types of cancerous or precancerous lesions. Markers or combinations of markers that are informative for early stage cancer or precancer are generally preferred, because patient prognosis is much better when cancer is detected early.

According to aspects of the invention, detecting the presence of an adenoma may be useful for detecting early signs of cancer or precancer. Adenomas are typically glandular tumors or tumors of glandular origin. Adenomas may be early indicia of cancer, for example colon cancer. Not all adenomas become cancers. However, many cancers (e.g., carcinomas such as colorectal carcinomas) are thought to develop from adenomas. Indeed, a majority of colon cancers are thought to develop from adenomas. Therefore, detecting adenomas is particularly useful for identifying early signs or risks of colorectal cancer (e.g., cancerous and precancerous lesions or growths in the colon).

Adenomas may be invasive adenocarcinomas, significant adenomas, or low potential polyps. Invasive adenocarcinomas may be, for example, adenocarcinomas at different TNM stages (e.g., TNM stages 1, 2, 3, or 4). Significant adenomas may be, for example, carcinomas in-situ/high-grade dysplasias (CIS/HGD) having a diameter of greater than 1 cm, about 1 cm, less than 1 cm, or of unknown size; vilous adenomas having a diameter of greater than 1 cm, about 1 cm, less than 1 cm, or of unknown size; tubulovillous adenomas having a diameter of greater than 1 cm, about 1 cm, less than 1 cm, or of unknown size, and low-grade dysplasias (LGD) with a diameter of greater than or equal to 1 cm. Low potential polyps may be, for example, advanced polyps, and adenoma low-grade dysplasias (LGD) with an unknown diameter or a diameter of less than 1 cm. Aspects of the invention may be useful to detect any one or more of these different types of adenomas.

According to aspects of the invention, adenomas can be detected at different positions in the colon and rectum (including the right and left colon and the transverse colon).

Aspects of the invention include panels of markers (e.g., genetic abnormalities such as mutations, including point mutations, deletions, and/or insertions) with different levels of informativeness for adenomas. According to the invention, a panel of markers may include a plurality of different markers, any one of which may be indicative of disease (e.g., the presence of an adenoma) if it is detected. The sensitivity level of a marker panel is related to the percentage of diseased individuals (e.g., individuals with an adenoma) that are positive for at least one marker in the marker panel. Accordingly, only one positive marker out of all the markers tested in a panel may be sufficient to detect an adenoma. However, an adenoma may be associated with the presence of two or more markers from the marker panel. It should be appreciated that different marker panels may have different levels of sensitivity. According to the invention, a marker may be a mutation (e.g., a point mutation, a deletion, an insertion, or other nucleic acid alteration) relative to a normal sequence at a defined nucleic acid position (e.g., genomic position) or within a defined region (e.g., a defined genomic region). Accordingly, one or more of the markers described herein may be a mutation (e.g., a sequence difference) relative to one or more of the sequences provided in SEQ ID NOs. 1-14 at the specific positions or regions provided herein for the marker(s) in the marker panel(s).

In one embodiment, the following panel may be used to detect adenomas with greater than 60% sensitivity: assays are performed to detect one or more genetic abnormalities from a multiple mutation panel of genetic abnormalities at 22 loci including Kras mutations in codon 12 (K12 position 1, K12 position 2) and codon 13 (K13 position 2); mutations in APC codons 1309 (deletions), 1306 (mutations at position 1), 1312 (mutations at position 1), 1367 (mutations at position 1), 1378 (mutations at position 1), 1379 (mutations at position 1), 1450 (mutations at position 1), 1465 (deletions), 876 (mutations at position 1) and 1554 (insertions); mutations in P53 codons 175 position 2, 245 position 1, 245 position 2, 248 position 1, 248 position 2, 273 position 1, 273 position 2 and 282 position 1; and deletions at the BAT-26 locus. This panel is referred to herein as the V1 panel. Mutations at these loci can be detected using primer extension assays (including single base extension assays and assays designed to detect deletions or insertions in the polyA tract of the BAT-26 locus) or other assays that are useful to detect one or more of these genetic abnormalities (e.g., scanning or base tracking for identifying one or more mutations within a target region rather than assaying for a mutation at one specific position). In certain embodiments, mutant specific hybridization assays may be used. In some embodiments, sequencing assays may be used. In certain embodiments, mutant specific amplification assays may be used. Non-limiting examples of assays that may be used to detect one or more point mutations, deletions, or insertions, include one or more assays disclosed in issued U.S. Pat. Nos. 6,280,947; 6,482,595; 6,503,718; or in U.S. patent publication 20030203382, the disclosures of which are incorporated herein. One or more oligonucleotides may be used to capture, amplify, and/or assay for one or more of these markers (e.g., mutations). Different oligonucleotides may be designed based on the known nucleic acid sequences of Kras, APC, P53, and BAT-26 (for example, oligonucleotides having sequences identical to, or complementary to, portions of SEQ ID NO: 2 or 12 may be used for Kras marker analysis; oligonucleotides having sequences identical to, or complementary to, portions of SEQ ID NO: 1 or 11 may be used for APC marker analysis; oligonucleotides having sequences identical to, or complementary to, portions of SEQ ID NO: 3 or 13 may be used for P53 analysis; and oligonucleotides having sequences identical to, or complementary to, portions of SEQ ID NO: 10 may be used for Bat-26 analysis). The annealing position and size of the oligonucleotide(s) may be determined in part by the type of assay that is used.

In another embodiment, the following panel may be used to detect adenomas with greater than 60% sensitivity: assays may be performed to detect hypermethylation at one or both of the HLTF locus and the V29 locus (a Vimentin locus). Hypermethylation at these loci can be detected using methylation specific primer analysis (e.g., MSP amplification) or other assays that are useful to detect hypermethylation at one or more of these genetic loci. Non-limiting examples of assays that may be used to detect hypermethylation at one or more loci include one or more assays disclosed in U.S. patent publications 20040053304; 20040242510; 20050106593; and in issued U.S. Pat. Nos. 5,786,146; 6,017,704; 6,200,756; 6,265,171; 6,818,404; and 6,960,436, the disclosures of which are incorporated herein.

In one embodiment, scanning for one or more mutations at the APC-MCR (the APC mutation cluster region, see for example Miyoshi et al., 1992, Hum. Mol. Genet. 1(4):229-33) may detect adenomas with greater than 74% sensitivity. Scanning may be performed using a base scanning technique described herein, or any other suitable detection assay, to scan for the presence of one or more mutations within the APC sequence, the APC-MCR, for example within the sequence of SEQ ID NO: 1 or 11 or any portion thereof.

In one embodiment, the following panel may be used to detect adenomas with greater than 90% sensitivity: scanning for one or more mutations in the APC-MCR locus, exon 9 of the PIK3CA locus, exon 20 of the PIK3CA locus, B-catenin (e.g., exon 5), or a mutation in BRAF that results in a V599E amino acid change. Scanning as described herein can be used to detect one or more mutations in the APC-MCR locus, exon 9 of the PIK3CA locus, or exon 20 of the PIK3CA locus. Mutations at the BRAF locus can be detected via primer extension or other appropriate methodology (including scanning). One or more oligonucleotides may be used to capture, amplify, and/or assay for one or more of these markers (e.g., mutations). Different oligonucleotides may be designed based on the known nucleic acid sequences of PIK3CA (see, for example, Samuels et al., 2004, Science 304(5670):554), B-catenin (see, for example, Sparks et al., 1998, Cancer Res. 58(6), 1130-4), and BRAF. For example, oligonucleotides having sequences identical to, or complementary to, portions of SEQ ID NO: 4 or 5 may be used for B-catenin marker analysis; oligonucleotides having sequences identical to, or complementary to, portions of SEQ ID NO: 6 or 14 may be used for BRAF marker analysis; and oligonucleotides having sequences identical to, or complementary to, portions of SEQ ID NO: 7, 8, or 9 may be used for PIK3CA analysis. The annealing position and size of the oligonucleotide(s) may be determined in part by the type of assay that is used.

In one embodiment, a combination of all of the above loci may be used to detect adenomas with a greater than 95% sensitivity (e.g., greater than 98% sensitivity).

Data obtained for 50 colonic adenomas that were analyzed using these different panels of markers is described in more detail in the examples.

It should be appreciated that other combinations of these markers can be used to obtain different levels of sensitivity for detecting adenomas (e.g., colonic adenomas).

Assays can be performed on stool samples (e.g., representative stool samples) using methods that can detect small amounts of mutant genetic material in a heterogeneous sample containing a majority of normal genetic material (e.g., where mutant genetic material accounts for less than 10%, less than 5%, less than 2.5%, and even less than 1% of the total genetic material in the sample). Accordingly, a genetic marker belonging to an informative panel of markers may be detected using one or more methods that can detect a low frequency event in a heterogeneous biological sample. Such methods may include PCR amplification, primer extension, and/or mutant scanning methods. The specificity and sensitivity of a primer extension or scanning reactions that include acyclo terminators and deoxynucleotides can be improved by using a combination of acyclo polymerase and TAQ polymerase (or other combination of a polymerase that preferentially incorporates acyclo terminators and a polymerase that preferentially incorporates deoxynucleotides). In some embodiments, one or more assays may be performed in a digital format (e.g., diluted so that on average 1, 1 to 5, or a few more different molecules are analyzed in each assay—it should be appreciated that the diluted sample may be amplified to increase the signal in each assay). In one embodiment, a digital analysis (e.g., a digital amplification and subsequent analysis) may be performed on at least a sufficient number of molecules to obtain a statistically significant result. Certain digital techniques are known in the art, see for example, U.S. Pat. No. 6,440,706 and U.S. Pat. No. 6,753,147, the entire contents of which are incorporated herein by reference. Similarly, an emulsion-based method for amplifying and/or sequencing individual nucleic acid molecules may be used (e.g., BEAMing technology).

In one embodiment, a sequencing method that can sequence single molecules in a biological sample may be used. Sequencing methods are known and being developed for high throughput (e.g., parallel) sequencing of complex genomes by sequencing a large number of single molecules (often having overlapping sequences) and compiling the information to obtain the sequence of an entire genome or a significant portion thereof. According to the invention, such methods, although designed for complex sequence analysis, may be particularly suited to sequence a large number of substantially identical molecules in order to identify the rare one(s) that contain a mutation or alteration.

High complexity analytical or sequencing techniques may involve high speed parallel molecular nucleic acid sequencing as described in PCT publication WO01/16375, U.S. application 60/151,580 and U.S. published application 20050014175, the entire contents of which are incorporated herein by reference. Other non-limiting sequencing techniques are described in PCT publications WO05/73410, WO05/54431, WO05/39389, WO05/03375, WO05/010145, WO04/069849, WO04/70005, WO04/69849, and WO04/70007, and U.S. published application 20050100932, the entire contents of which are incorporated herein by reference.

High complexity analytical or sequencing techniques may involve exposing a nucleic acid molecule to an oligonucleotide primer and a polymerase in the presence of a mixture of nucleotides. Changes in the fluorescence of individual nucleic acid molecules in response to polymerase activity may be detected and recorded. The specific labels attached to each nucleic acid and/or nucleotide may provide an emission spectrum allowing for the detection of sequence information for individual template nucleic acid molecules. In certain embodiments, a label is attached to the primer/template and a different label is attached to each type of nucleotide (e.g., A, T/U, C, or G). Each label emits a distinct signal which is distinguished from the other labels.

High complexity analytical or sequencing techniques may involve or be based on methods or technology described in Shendure et al., Nature Reviews/Genetics, Volume 5, May 2004, pages 335-344; Braslavsky et al., PNAS, Apr. 1, 2003, Volume 100, No. 7, pages 3960-3964; the entire disclosures of which are incorporated herein by reference.

In other embodiments, high complexity analytical or sequencing techniques may involve providing a primed target polynucleotide linked to a microfabricated synthesis channel, and flowing a first nucleotide through the synthesis channel under conditions such as to allow the first nucleotide to attach to the primer. The presence or absence of a signal is determined, the presence indicating that the first nucleotide was incorporated into the primer and the identity of the complementary base that served as a template in the target polynucleotide is determined. The signal is then removed or reduced and the process repeated with a second nucleotide. The second nucleotide can be either the same as the first nucleotide or a different nucleotide. The specific labels attached to each nucleic acid provide an emission spectra allowing for detection of sequence information of the nucleic acid molecule. In other embodiments, a plurality of different primed target polynucleotides linked to different synthesis channels may be used. In further embodiments, the polynucleotide may be attached to a surface. In some embodiments, a label is attached to the nucleotide.

In certain embodiments, a high complexity analytical or sequencing technique may be provided by Helicos BioSciences Corporation (Cambridge, Mass.). In some embodiments, a nucleic acid polymerase and a fluorescently labeled nucleotide may be added to an assay to bind to immobilized templates (e.g., bound to appropriate primers). The sample may be washed to remove unbound nucleotides and excess polymerase. The sample may be analyzed and the positions of the incorporated nucleotides recorded. The fluorescent label may be removed and a second labeled nucleotide may be added. The process may be repeated several times until a desired length is reached.

Other useful genome/complex sequencing methods include high throughput sequencing using the 454 Life Sciences Instrument System. Briefly, a sample of single stranded DNA may be prepared and added to an excess of DNA capture beads which are then emulsified. Clonal amplification may be performed to produce a sample of enriched DNA on the capture beads (the beads are enriched with millions of copies of a single clonal fragment). The DNA enriched beads may be then transferred into PicoTiterPlate™ and enzyme beads and sequencing reagents may be added. The samples may be analyzed and the sequence data recorded. Pyrophosphate and luciferin are examples of the labels that can be used to generate the signal.

In other embodiments, single molecule sequencing technology available from US Genomics, Mass., may be used. For example, technology described, at least in part, in one or more of U.S. Pat. Nos. 6,790,671; 6,772,070; 6,762,059; 6,696,022; 6,403,311; 6,355,420; 6,263,286; and 6,210,896, the disclosures of which are incorporated herein may be used.

Similar assays can be performed on other heterogeneous biological samples including fluids and mucus (e.g., urine, blood, serum, sputum, semen, breast nipple aspirate, or other bodily fluids).

Assays also may be performed on one or more tissue biopsies (e.g., colon biopsies or biopsies of other tissues or organs). Tissue biopsies are expected to contain more abnormal genetic material if they are positive for an adenoma. Accordingly, assays performed on tissue biopsies may not be as specific and sensitive as assays performed on heterogeneous biological samples.

Regardless of the source of nucleic acid (e.g., a biological sample, a tissue biopsy, etc.), an analysis may involve a nucleic acid capture step, a nucleic acid amplification step, and/or a nucleic acid analysis step (e.g., a using mutation-specific detection technique or a scanning technique, etc.). A capture probe that is complementary to one of the strands in the region of a sequence being assayed (e.g., the locus or position being interrogated for the presence of one or more point mutations, deletions, insertions, etc., or any combination thereof) may be used to capture nucleic acid fragments for subsequent analysis. A capture probe may be between about 20 and about 50 nucleotides long (e.g., between about 25 and about 45, or about 30, about 35, or about 40 nucleotides long). However, in some embodiments shorter or longer capture probes may be used. A capture probe may be designed to be complementary to a sequence that is found in the vicinity of the region being assayed (e.g., within about 10, 20, 30, 40, 50, 60, 70, 80, 90, 100, 150, 200, 250, 500, or 1,000 nucleotides or more of the nucleic acid region being analyzed). It should be appreciated that in some embodiments, the distance between the capture probe binding site and the region of interest may be determined in part by the size of the fragment that is being amplified after capture. However, in many embodiments, a capture probe is more effective when it is close to a sequence being assayed. Accordingly, a capture probe may be identical or complementary to one or more sequences of any of SEQ ID NO: 1-14. A capture probe may be attached to a solid support and used to hybrid capture sample nucleic acid of interest (e.g., via electrophoresis, repeated electrophoresis, chromatography, repeated chromatography, mixing, etc.)

In some embodiments, a captured nucleic acid may be amplified. For example, a PCR amplification may be performed using a pair of amplification primers designed to amplify a region containing the sequence being assayed. In some embodiments, the amplification primers may amplify the region that hybridized to the capture probe. In some embodiments, the amplified region may be adjacent to (but does not include) the sequence that was bound during hybrid capture. Amplification primers may be between about 10 and about 50 nucleotides long (e.g., between about 15 and about 45, or about 20, about 25, about 30, about 35, about 40, or about 45 nucleotides long). However, in some embodiments shorter or longer amplification primers may be used. It should be appreciated that each primer in a pair of amplification primers may be complementary to a different (complementary) strand of the nucleic acid region being amplified. Accordingly, an amplification primer may be identical or complementary to one or more sequences of any of SEQ ID NO: 1-14. The amplification primers may be designed to amplify regions of different sizes. In some embodiments, amplification products may be from about 30 to about 5,000 nucleotides long (e.g., about 40, 50, 75, 100, 150, 200, 250, 500, 750, or 1,000 or more) depending, in part, on the assay format and the number and spacing of markers that are to be analyzed on the single amplification product. However, amplification products of shorter, longer, or intermediate sizes also may be analyzed. In some embodiments, one or more amplification products may be used to assay multiple positions on a predetermined gene or genetic region (e.g., within the APC, Kras, p53 or other locus). In some embodiments, a separate amplified region may be assayed for each marker in the panel. In some embodiments, several markers (e.g., 2, 3, 4, 5, 5-10, or more) may be assayed on a single amplified nucleic acid. In some embodiments, one or both amplification primers may be methylation specific primers (primers that are specific for methylated C by having Gs to pair with methylated C that is not modified by bisulfite treatment in a methylation detection assay).

Oligonucleotide primers also may be used for marker detection (e.g., on an amplified nucleic acid). Primers may be used for many different hybridization and/or extension based assays (e.g., one or more extension assays, single base extension assays, sequencing assays, scanning assays, methylation detection assays, etc.). Assay primers may be between about 10 and about 50 nucleotides long (e.g., between about 15 and about 45, or about 20, about 25, about 30, about 35, about 40, or about 45 nucleotides long). However, in some embodiments shorter or longer amplification primers may be used. An assay primer may be designed to anneal to a target region adjacent or near a site of interest. For example, the 3′ end of an assay primer may anneal immediately upstream of a position opposite a target position that is suspected of being altered (e.g., mutant) on the nucleic acid that is being assayed. In some embodiments, the 3′ end of the assay primer may anneal 1 or more nucleotides upstream from the target position (e.g., 1, 2, 3, 4, 5, 6, 7, 8, 9, 10, 10-15, 15-20, 20-25, 25-50, 50-100, or more nucleotides upstream from the target position). It should be appreciated that the presence of a marker (e.g., a mutation) may be assayed on either strand of a nucleic acid region of interest using appropriate assay primers. In some embodiments, an assay primer may be methylation specific.

Accordingly, in certain embodiments, a specific hybrid capture method may involve using a capture probe to bind to a target nucleic acid. The bound product then may be isolated. In one embodiment, a capture probe may be bound to a solid surface thereby acting as an anchor for isolating a target molecule. In other embodiments, a capture probe may be modified in a manner that allows it to be isolated or purified from a sample. For example, a capture probe may biotinylated, attached to an antigen, attached to a magnetic particle, attached to a molecular weight marker, attached to a charged particle, attached to another particle or other molecular “hook” that can be used to isolate that capture probe and thereby isolate a target molecule that is hybridized to the probe.

In aspects of the invention, a nucleic acid preparation may be captured by repeated exposure of a biological sample (for example, a processed biological sample) to a capture probe on a solid support or in a medium, for example, by the rapid flow of the sample past a capture probe for the target nucleic acid molecule. The repetitive nature of such a method allows for a target molecule to bind and enhances the total number of molecules bound to the capture probe, providing a high yield capture. The solid support may be an electrophoretic medium (e.g., gel or beads) and the repetitive exposure of the sample to the capture probe may involve exposure to repeated cycles of electrophoresis in alternate directions (back and forth across a solid support region containing one or more different types of capture probe). In some aspects, a sample is added to a portion of an electrophoretic medium having at least two regions arranged consecutively in a first spatial dimension. In some aspects, at least one of the at least two regions includes a first capture probe which is immobilized within that region. An electric field is applied to the electrophoretic medium in a first direction which is parallel to the first dimension. An electric field is then applied to the electrophoretic medium in a second direction which is opposite to the first direction. In further aspects, the electric field is applied repeatedly in each direction. For further details see for example U.S. published application 20050247563 or PCT publication WO2005/047881, the entire contents of which are incorporated herein by reference.

In aspects of the invention, a sample may be a biological sample. A biological sample may be, but is not limited to, stool, whole blood, serum, plasma, tears, saliva, nasal fluid, sputum, ear fluid, genital fluid, breast fluid, milk, colostrum, placental fluid, amniotic fluid, perspirate, synovial fluid, ascites fluid, cerebrospinal fluid, bile, gastric fluid, aqueous humor, vitreous humor, gastrointestinal fluid, exudate, transudate, pleural fluid, pericardial fluid, semen, upper airway fluid, peritoneal fluid, fluid harvested from a site of an immune response, fluid harvested from a pooled collection site, bronchial lavage, urine, biopsy material, a nucleated cell sample, a fluid associated with a mucosal surface, hair, or skin. A sample also may be a pooled sample containing biological material and or isolated nucleic acids from a plurality of subjects (e.g., 2, 3, 4, 5, about 10, or more).

In aspects of the invention, a large amount of sample may be processed in order to increase the confidence level of isolating or capturing a rare event indicative of very early stage disease (e.g., an adenoma, an early stage cancer, etc.). For example, about 10 g, about 20 g, about 30 g, about 40 g, about 50 g, about 60 g, about 70 g, about 80 g, about 90 g, about 100 g, about 150 g, about 200 g, or more stool sample may be processed using a capture technique described herein.

In embodiments of the invention, exposure of a biological sample (for example a crude preparation of total nucleic acid from a biological sample) to immobilized capture probe(s) may be repeated between 2 and 100 times, e.g., between about 5 and about 50 times, between about 10 and about 40 times, or about 11, 12, 13, 14, 15, 16, 17, 18, 19, 20, etc. times, including about 25, 30, or 35 times.

In aspects of the invention, a sample may be exposed repeatedly to a capture probe using chromatographic methods, for example high performance liquid chromatography (HPLC), fast performance liquid chromatography (FPLC), etc., or any combination thereof.

A captured preparation of target nucleic acid molecules (e.g., of low genomic complexity) may be eluted using any suitable technique and prepared (e.g., single stranded molecules may be prepared) for subsequent analysis using a technique for analyzing nucleic acid samples of high genomic complexity. Sample capture techniques described herein may be used to analyze DNA and/or RNA.

Methods of the invention may be useful for screening an individual as part of a routine cancer screen. Methods of the invention may be useful as part of a population screen to identify individuals with early stages of cancer (e.g., colon cancer). Methods of the invention may be used to test patients suspected of having colon cancer (e.g., patients with polyps, a family history of colon cancer, or other indicators of cancer such as blood in the stool, etc.). Polyps may include non neoplastic polyps with a diameter of 1 cm or more (potentially significant polyps) and non neoplastic polyps with a diameter of less than 1 cm or an unknown diameter.

In certain embodiments, a general population screen may be performed with markers that are greater than 60% informative. For example, a general population screen may be performed using a panel of multiple mutations (e.g., a multiple mutation panel described herein). In another example, a general population screen may be performed using an assay for hypermethylation. In yet a further embodiment, a general population screen may be based on a scanning assay. Of course, any combination of the above types of assays may be used to obtain higher informativeness.

Analyses of individual patients may be performed using assays of different informativeness. If a patient has one or more signs of colon cancer (e.g., blood in the stool, a history of polyps, etc.) or other risk factors (e.g., age, diet, exposure to carcinogens) an analysis of a marker panel that is sufficient for obtaining a sensitivity of 90%, 95% or more may be recommended. In other embodiments, a screen of a subset of markers (e.g., a panel of markers with lower informativeness) may be recommended for a general health screen of younger individuals (e.g., younger than 50, 40, 30, 20 etc.) with no other risk factors or indicia of cancer.

Accordingly, aspects of the invention may be useful for detecting adenomas with greater than about 60%, greater than about 70%, greater than about 80%, greater than about 90%, or greater than about 95% confidence. Therefore, aspects of the invention are useful for assaying patient samples for adenomas and reducing the number of false negatives to fewer than about 40%, fewer than about 30%, fewer than about 20%, fewer than about 10%, or fewer than about 5%.

In one aspect, an assay may be performed on a regular basis (e.g., annual medical checkup). The informativeness of this assay may increase over time, because additional genetic markers may become positive as adenomas develop over time. Therefore, initial false negatives may show up as positives in subsequent genetic tests. These later identifications still be may early enough for the patient to have a positive prognosis.

In general, the detection of one or more indicators of adenomas in a screen of the invention may be followed up by one or more subsequent analyses to locate and/or confirm the presence of an adenoma, and ultimately to treat any cancerous or precancerous lesions that may be detected. Invasive procedures such as colonoscopies or sigmoidoscopies may be used to locate and sample tissue for further analysis. Other less invasive procedures may include virtual colonoscopies. Treatments may include surgical removal (e.g., of the lesion or of a region of the colon or other organ or tissue that contains the lesion), radiation, chemotherapy, or any combination thereof.

It should be appreciated that aspects of the invention described herein in the context of colonic adenomas and colon cancer may be used to screen patient samples for the presence of adenoma in other tissues.

According to aspects of the invention, biopsies that are removed in order to identify the source of mutant genetic material may be assayed using methods described herein. As discussed above, it should be appreciated that different cutoff levels may be used for tissue biopsy samples than for non-tissue samples, because biopsy samples may contain relatively more adenoma cells than biological fluid or solid samples (e.g., mucus, stool, etc.).

In another aspect, the invention provides groups of nucleic acid probes or primers that are useful for assaying a biological sample for the presence of each genetic abnormality included in a genetic abnormality panel that is at least 60% informative for adenoma. Certain nucleic acid probes or primers may be useful for amplifying regions of the genome that contain one or more genetic loci of interest. Certain nucleic acid probes or primers may be useful for performing primer extension reactions on amplified or non-amplified template nucleic acid in order to assay for the presence of one or more genetic abnormalities. Primers may be oligonucleotides ranging from about 10 nucleotides to about 100 nucleotides in length, and preferably from about 20 to about 50 nucleotides in length. A primer for a single base extension reaction may be complementary to a genomic sequence that is adjacent to the position of a genetic abnormality included in an informative panel of genetic markers. Accordingly, aspects of the invention include a panel or group of oligonucleotides designed to interrogate a biological sample for the presence of each genetic abnormality that belongs to a panel that is at least about 60% informative for adenoma (and preferably at least about 70%, at least about 80%, at least about 90%, or at least about 95% informative for adenoma). Accordingly, aspects of the invention include a panel of assays designed to interrogate each of a panel of genetic loci for the presence of a genetic abnormality indicative of adenoma. Therefore, a kit of the invention may include one or more of a capture probe, an amplification primer pair, and/or an extension primer (or any combination of two or more thereof) for each locus that is being analyzed. In some embodiments, the kit may include a capture probe and/or an amplification primer pair and/or an extension primer for each assay included in a panel of assays described herein. It should be appreciated that in some embodiments, a single capture probe and/or a single amplification primer pair may be used to capture and/or amplify a single region that may be assayed for two or more markers (e.g., 2, 3, 4, 5, 6, 7, 8, 9, 10, 11, 12, 12-20 or more). For example, a single Kras region may be captured and/or amplified and subsequently assayed for mutations at two or more of the positions that are included in a chosen panel of markers.

### EXAMPLES

### Example 1

A commercially available 22 marker multiple mutation panel (V1) was developed for the detection of colorectal carcinomas and analysis of 147 cancer tissues revealed informativeness of ˜71%. V2 markers including two hypermethylation sites, mutation site BRAF and scanning sites APC-MCR, PIK3CA-Ex9 and Ex20 and B-catenin were tested in the same tissues with resulting informativeness of 88% and an overall informativeness (i.e., V1+V2) of ˜97%. To determine informativeness in adenoma, fifty tissue samples were tested with all V1 and V2 markers.

Results for V1 and V2 markers in 50 adenoma tissues are shown in Table 1. Two V1 cutoffs are presented that represent cutoffs based on stool data (Cutoff 1) and more conservative cutoffs based on tissues that were>2 fold over stool cutoffs (Cutoff 2). The APC-MCR scan had the highest informative value of 74% and V2 markers alone detected 94% of the adenomas suggesting this would be a good panel for early stage CRC detection.

TABLE 1

| | Cutoff 1 | Cutoff 2 |
|---|---|---|
| V1 | 62.0% | 54.0% |
| V2 | 94.0% | 94.0% |
| V1 + V2 | 98.0% | 94.0% |
| | Alg-1A | Ig-2 |
| KRAS | 38.0% | 32.0% |
| APC | 38.0% | 34.0% |
| P53 | 4.0% | 2.0% |
| BAT-26 | 4.0% | 4.0% |
| Total MuMu | 62.0% | 54.0% |
| APC-MCR | 74.0% | |
| PIK3CA-9 | 14.0% | |
| PIK3CA-20 | 4.0% | |
| B-CAT | 4.0% | |
| BRAF | 4.0% | |
| Total Scan | 90.0% | |
| HLTF | 38.0% | |
| V29 | 50.0% | |
| Total Methylation | 60.0% | |

### Example 2

### Scanning (Base Tracking)

Scanning or base tracking methods of the invention may be used to screen a nucleic acid region (e.g., the APC-MCR) for the presence of one or more mutations at different positions within the nucleic acid region. Current methods of nucleotide sequencing use a single sequencing reaction containing a mixture of all four terminator nucleotides in the same reaction, where each terminator base is differentially labeled and detected. The signal from an altered sequence present at low concentrations in a sequencing reaction is often masked by the signal of the wild type base at the same location. Variant sequences must be at least about 10% of the DNA being sequenced before their presence can be readily detected. In contrast, methods according to the invention increase the sensitivity of assays to detect nucleic acid alterations that are present at a relatively low level in a sample, especially, e.g., in a heterogeneous sample.

The present invention includes methods of screening nucleic acids for at least one genetic variation through the application of a novel modification of a DNA sequencing reaction. Methods of the invention modify current sequence reactions such that only one terminator nucleotide, and not all four terminator nucleotides, is provided in the primer extension reaction to allow for single base scanning, which is also referred to herein as single base tracking. The modified reaction is herein referred to as a single base tracking reaction.

Sensitivity is increased in single base tracking at least because signals from bases at any one position in a sequence being scanned are no longer masked by signals from an alternate base in the wild type sequences present at higher concentrations in the sample. Therefore, methods of the invention detect the presence of nucleotide sequences with altered residues as compared to a control “wild type” nucleotide sequence, where the nucleotides with altered sequence make up about 50%, about 25%, about 10%, about 5%, about 4%, about 3%, about 2.5%, about 2%, about 1.5% or especially about 1% of the sample being analyzed. Such an increase in sensitivity has at least several uses. For example, methods according to the invention can be used to screen the human genome, providing for increased sensitivity for detection of low frequency genetic variations.

In a preferred reaction, the terminator nucleotide is labeled. A preferred label is a fluorescent label, although it is within the skill of an artisan to use substitute labels of equal or higher sensitivity in signal detection, and/or equal or lower background signal noise. The DNA single base tracking reaction utilizes sensitive labeling techniques in order that the resulting sequence fragments may be analyzed and, e.g., compared to a known normal control sample to determine whether at least one genetic variation exists between the sample and normal control.

Additionally, methods of the invention can be used to screen for mutations that are predicative of a disease state. Often, these mutations are present in a sample at a relatively low level, e.g., where the mutation is a somatic mutation in a nucleic acid population obtained from biopsied tissue. Accordingly, methods according to the invention are more sensitive than current methods and can detect relatively low frequency mutations in a heterogeneous sample.

One aspect of the invention includes a method for detecting a difference between two nucleic acids. The method includes extending a first primer complementary to a target nucleic acid in the presence of a first nucleotide and a second nucleotide to produce at least one product. The first nucleotide is at least one deoxynucleotide, and more preferably is a mixture of the four deoxynucleotides, namely dATP, dCTP, dGTP and dTTP (“dNTP mixture”), used for the elongation step of the primer extension reaction. The second nucleotide is a terminator nucleotide, preferably includes a detectable label, and has the same base as one of the first deoxynucleotides. The method also includes detecting a signal from the at least one product and comparing the signal from the at least one product with a signal that is generated from a comparison nucleic acid in substantially the same manner as the signal is generated from the target nucleic acid. A difference between the signals indicates at least one difference between the target nucleic acid and the comparison nucleic acid. Signal differences include the addition of at least one peak, the deletion of at least one peak, or a shift in the position of at least one peak present in the sample as compared to the control.

The embodiments described above and below can have any or all of the following features. The method may include the step of amplifying a nucleic acid to form the target nucleic acid. The extending step can include extending the primer in the presence of the deoxynucleotides dATP, dCTP, dGTP, and dTTP. The target nucleic acid can be a nucleic acid suspected of containing a mutation. The target nucleotides to be screened in the methods of the invention may be genomic DNA, complementary DNA (cDNA), or RNA. Where the initial sample is RNA, it is preferred that the RNA is converted into DNA prior to further processing. The extending and comparing steps can be repeated. The extending and comparing steps can be conducted at least four times with the same primer, each time using a different one of adenine (A), cytosine (C), guanine (G) or thymidine (T) for the base of the second “terminating” nucleotide (i.e., each extension reaction contains only one type of extension terminating nucleotide, where the terminating nucleotide may be a dideoxynucleotide or an acyclonucleotide, and the base of the terminating nucleotide is chosen from A, C, G, or T.

The comparison nucleic acid can be a wild type nucleic acid. The signal from the comparison nucleic acid can be determined prior to, at the same time as, or after the signal from the target nucleic acid. The signal can include a fluorescent light emission. Alternatively, the signal results of the control sequence may be obtained from a database of nucleotide sequences. The comparison step may be done manually or by automation.

The methods described above or below can also have any or all of the following features. In certain embodiments, the method includes extending a second primer complementary to the target nucleic acid in the presence of the first nucleotide and the second nucleotide to produce at least one secondary product. In a preferred embodiment, the first nucleotide is a deoxynucleotide (dNTP) mixture, the second nucleotide is a terminator nucleotide (dideoxynucleotide or acyclonucleotide) of only one base selected from A, C, G or T, and the at least one secondary product is the product of a primer extension reaction. The method may also include detecting a signal from the at least one secondary product and comparing the signal from the at least one secondary product with a signal that was generated from a comparison nucleic acid in substantially the same manner as the signal was generated from the target nucleic acid. A difference between the signals indicates at least one difference between the target nucleic acid and the comparison nucleic acid.

The methods described above or below may also include the following features. In one embodiment, a second primer complementary to a strand complementary to the target nucleic acid is extended in the presence of the first nucleotide and the second nucleotide to produce at least one secondary product. In a preferred embodiment, the first nucleotide is a deoxynucleotide (dNTP) mixture, the second nucleotide is a terminator nucleotide (dideoxynucleotide or acyclonucleotide) of only one base selected from A, C, G or T, and the at least one secondary product is the product of a primer extension reaction. The method can then include detecting a signal from the at least one secondary product and comparing the signal from the at least one secondary product with a signal that is generated from a comparison nucleic acid in substantially the same manner as the signal is generated from the target nucleic acid. A difference between the signals indicates at least one difference between the target nucleic acid and the comparison nucleic acid.

In another aspect of the invention, a method for detecting a difference between two nucleic acids includes extending a first primer complementary to a target nucleic acid in the presence of a first nucleotide including a detectable label and a second nucleotide to produce at least one product. In a preferred embodiment, the first nucleotide is a deoxynucleotide (dNTP) mixture, the second nucleotide is a terminator nucleotide (dideoxynucleotide or acyclonucleotide) of only one base selected from A, C, G or T, and the at least one product is the product of a primer extension reaction. The method also includes detecting a signal from the at least one product and comparing the signal from the at least one product with a signal that is generated from a comparison nucleic acid in substantially the same manner as the signal is generated from the target nucleic acid. A difference between the signals indicates at least one difference between the target nucleic acid and the comparison nucleic acid.

In another aspect of the invention, a method for detecting a difference between two nucleic acids includes extending a first primer including a detectable label and being complementary to a target nucleic acid in the presence of a first nucleotide and a second nucleotide to produce at least one product. In a preferred embodiment, the first nucleotide is a deoxynucleotide (dNTP) mixture, the second nucleotide is a terminator nucleotide (dideoxynucleotide or acyclonucleotide) of only one base selected from A, C, G or T, and the at least one product is the product of a primer extension reaction. The method also includes detecting a signal from the at least one product and comparing the signal from the at least one product with a signal that was generated from a comparison nucleic acid in substantially the same manner as the signal was generated from the target nucleic acid. A difference between the signals indicates at least one difference between the target nucleic acid and the comparison nucleic acid.

In another aspect of the invention, a method for detecting a difference between two nucleic acids includes extending a first primer complementary to a target nucleic acid in the presence of a first nucleotide and a second nucleotide to produce at least one product. The second nucleotide is a terminator nucleotide and includes the same base as the first nucleotide. In a preferred embodiment, the first nucleotide is a deoxynucleotide (dNTP) mixture, the second nucleotide is a terminator nucleotide (dideoxynucleotide or acyclonucleotide) of only one base selected from A, C, G or T, and the at least one product is the product of a primer extension reaction. The method also includes detecting a mass of the at least one product and comparing the mass of the at least one product with a mass that is generated from a comparison nucleic acid in substantially the same manner as the mass is generated from the target nucleic acid. A difference between the masses indicates at least one difference between the target nucleic acid and the comparison nucleic acid.

### Example 3

### Stool Sample Preparation

The following example illustrates a method for preparing a DNA sample from a stool sample, see for example U.S. published applications 2004-0043467 and 2004-0014104, the entire contents of which are incorporated herein by reference.

A stool sample is collected and may be stored at −80° C. before use. The sample is thawed and resuspended in buffer, for example 10 mM Tris-Cl pH 8.0, 1 mM EDTA and 150 mM NaCl, or other suitable buffer as known to those of ordinary skill in the art. In one embodiment, the buffer may contain between 100 mM and 200 mM EDTA, for example about 150 mM EDTA. A suitable ratio of buffer to sample may be used, for example between 5:1 and 20:1 (mls/g of sample), for example about 7:1. The sample is then homogenized utilizing an EXACTOR stool shaker (EXACT Laboratories Marlborough, Mass.). Following homogenization, the stool sample is centrifuged to remove all particulate matter, and the supernatants are incubated at 37° C. Proteinase K (0.5 μg/μL) and SDS (0.5%) may be added at this point. The DNA is extracted from the supernatant using Tris saturated phenol (Gibco/BRL, Grand Island, N.Y.), phenol/chloroform/isoamyl alcohol (25:24:1), and chloroform. The DNA is then precipitated (1/10 volume 3M NaAc and an equal volume isopropanol), removed from solution by centrifugation, and resuspended in TE (0.01M Tris pH 7.4, 0.001M EDTA) buffer containing RNase A (2.5 μg/mL), or other suitable buffer.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

All publications, patents and sequence database entries mentioned herein are hereby incorporated by reference in their entirety as if each individual publication or patent was specifically and individually indicated to be incorporated by reference. In case of conflict, the present application, including any definitions herein, will control.

1. A method of screening a subject for the presence of an adenoma, the method comprising
interrogating a biological sample obtained from a subject suspected of having an adenoma for the presence of each of a panel of genetic markers, wherein the panel of genetic markers is more than 60% informative for adenoma, and wherein the presence of one or more of the genetic markers in the biological sample is indicative of an adenoma in the subject.

2. The method of claim 1, wherein the panel of genetic markers is more than 70% informative.

3. The method of claim 1, wherein the panel of genetic markers is more than 80% informative.

4. The method of claim 1, wherein the panel of genetic markers is more than 90% informative.

5. The method of claim 1, wherein the panel of genetic markers is more than 95% informative.

6. The method of claim 1, wherein the panel of genetic markers comprises a hypermethylated genetic locus.

7. The method of claim 6, wherein the biological sample is interrogated for the presence of the hypermethylated genetic locus using a methylation specific primer extension or amplification assay.

8. The method of claim 6, wherein the hypermethylated genetic locus is the HLTF or V29 locus.

9. The method of claim 1, wherein the panel of genetic markers comprises a predetermined mutation in a cancer-associated genetic locus.

10. The method of claim 9, wherein the biological sample is interrogated for the presence of the predetermined mutation using a primer extension assay.

11. The method of claim 9, wherein the predetermined mutation is a mutation at the KRAS, APC, P53, BAT-26 or BRAF genetic locus.

12. The method of claim 1, wherein the panel of genetic markers comprises a mutation in a mutation cluster region.

13. The method of claim 12, wherein the biological sample is interrogated for the presence of a mutation in the mutation cluster region using a scanning assay.

14. The method of claim 6, wherein the mutation cluster region is APC-MCR, exon 9 of PIK3CA, exon 20 of PIK3CA, or an exon of B-catenin.

15. The method of claim 1, wherein the panel of genetic markers comprises a mutation in a mutation cluster region, a predetermined mutation in a cancer-associated genetic locus, and a hypermethylated genetic locus.

16. The method of claim 1, wherein the biological sample is a stool sample.

17. The method of claim 1, wherein the biological sample is a tissue biopsy sample.

18. The method of claim 1, wherein the adenoma is a colonic adenoma.

19. The method of claim 1, wherein the adenoma is an invasive adenoma.

20. A method of detecting indicia of adenoma in a biological sample, the method comprising assaying the biological sample for the presence of one or more genetic abnormalities from a group of genetic abnormalities that is more than 60% informative for adenoma.

21. A method of detecting adenoma in a subject, the method comprising performing, on a biological sample obtained from a subject suspected of having an adenoma, an assay that is more than 60% informative for adenoma.

22. A kit comprising a group of oligonucleotides, wherein each oligonucleotide is adapted for interrogating a genetic locus for the presence of a genetic marker belonging to panel that is at least 60% informative for adenoma.
